(19) United States
(12) Patent Application Publication
(10) Pub. No.: US 2011/0236314 A1
(43) Pub. Date: Sep. 29, 2011

(54) METHOD OF DETECTION AND DIAGNOSIS OF ORAL AND NASOPHARYNGEAL CANCERS

(75) Inventors: Soo-Hwang Teo, Subang Jaya (MY); Sok Ching Cheong, Subang Jaya (MY); Chai Phei Gan, Subang Jaya (MY); Rosnah Mohd Zain, Subang Jaya (MY)

(73) Assignee: Cancer Research Initiatives Foundation, Subang Jaya, Selangor Darul Ehsan (MY)

(21) Appl. No.: 13/063,710

(22) PCT Filed: Sep. 8, 2009
(86) PCT No.: PCT/MY09/00140

§ 371 Date: May 18, 2011

(30) Foreign Application Priority Data
Sep. 12, 2008 (MY) PI20083548

(51) Int. Cl. A61K 49/00; C40B 40/06; C12Q 1/68; G01N 33/68; C12Q 1/02; C07H 21/00; A61K 39/00; C07K 14/47; A61K 31/7088; A61K 39/395; C07K 16/18; C07H 21/02; A61P 35/00

(52) U.S. Cl. 424/9.1; 506/16; 435/6.12; 435/7.92; 435/29; 536/23.1; 424/277.1; 530/350; 536/23.5; 514/44 A; 424/174.1; 530/389.7; 536/24.5; 424/178.1; 435/6.1

(57) ABSTRACT

The present invention relates to cancer and in particular to oral and nasopharyngeal cancers. In particular, the present invention relates to a method of detection and diagnosis of oral squamous cell carcinoma (OSCC) and nasopharyngeal cancers by determining the expression levels of certain genes. The method comprising (a) determining in a biological sample from the patient the amount of the expression level of at least one gene selected from the group consisting of GNA-12 and IFITM3; and (b) comparing the determined expression levels of said genes in said biological sample with the level in a reference. The invention also relates to polypeptides, antibodies and nucleic acids of the invention for use in medicine and a kit for performing the invention.

### CROSS REFERENCE TO RELATED APPLICATIONS

This application is a national stage of international application no. PCT/MY2009/000140 filed Sep. 8, 2009, which claims priority from Malaysian patent application no. P120083548 filed Sep. 12, 2008, the contents of all of which are incorporated herein by reference in their entirety.

### FIELD OF THE INVENTION

The present invention relates to cancer and in particular to oral and nasopharyngeal cancers. In particular, the present invention relates to a method of detection and diagnosis of oral squamous cell carcinoma (OSCC) and nasopharyngeal cancers by determining the expression levels of certain genes. The invention also relates to polypeptides, antibodies and nucleic acids of the invention for use in medicine and a kit for performing the invention.

### BACKGROUND OF THE INVENTION

Oral cancer is a debilitating disease that is the 8^{th} and 13^{th} most common malignancy worldwide for males and females respectively (Parkin et al, 2005). It is estimated that up to 80% of these cancers occur in Asia. Although the epidemiology of oral cancer is well established, the prognosis and survival rates for oral cancer patients have not improved significantly over the past three decades (Mork, 1998). The main aetiological factors associated with this disease in the West are tobacco smoking and alcohol consumption, but in many Southeast Asian countries, similar lesions are closely associated with betel quid (BQ) chewing. BQ commonly used by individuals in these countries vary in their content but commonly include areca nut (ripe or unripe), betel leaf, lime and sometimes tobacco. Early work suggested that the spectrum of gene mutations in oral cancer tissues differ between Caucasian and Asian patients (Paterson et al, 1996; Saranath et al, 1991; Yeudall et al, 1993) suggesting that these genetic differences reflected the exposure to different risk factors and/or the social habits of the individual populations. This proposal is supported by recent in vitro experiments demonstrating that carcinogens that cause distinct gene mutations lead to the inactivation of different DNA repair and metabolic pathways (Bardelli et al, 2001). This has important implications, as these genetic changes may serve as molecular markers which can form the basis for future diagnostic and therapeutic strategies.

Although it has been demonstrated that the spectrum of genetic alterations differ between smokers and BQ chewers, many of these investigations focused on a relatively small number of individual genes whilst others analysed a number of molecules within a single pathway (Bradley et al, 2001; Lim at al, 2005; Thongsuksai et al, 2003). Genome-wide studies to investigate the differences between smoking and BQ associated cancers have hitherto not been conducted.

Thus, there still exists a need to understand the aetiology of oral squamous cell carcinoma (OSCC) between Western and Asian countries. More particularly, there exists a need for a method for aiding assessment of a patient's risk of developing oral and nasopharyngeal cancers, or likely severity or likelihood of progression of oral and nasopharyngeal cancers, or aiding in selection of a cancer treatment regime for the patient in treating oral and nasopharyngeal cancers, or aiding in assessment of a cancer treatment regime for treating oral and nasopharyngeal cancers.

### SUMMARY OF THE INVENTION

In accordance with a first aspect of the invention, there is provided a method for aiding assessment of a patient's risk of developing oral and nasopharyngeal cancers, or likely severity or likelihood of progression of oral and nasopharyngeal cancers, or aiding in selection of a cancer treatment regime for the patient in treating oral and nasopharyngeal cancers, or aiding in assessment of a cancer treatment regime for treating oral and nasopharyngeal cancers, or the detection and diagnosis of oral and nasopharyngeal cancers, the method comprising:

(a) determining in a biological sample from the patient the amount of the expression level of GNA-12 (Guanine Nucleotide Binding Protein Alpha 12); and

(b) comparing the determined expression levels of said gene in said biological sample with the level in a reference.

In the preferred embodiment of the present invention, step (a) further comprising the step of determining the amount of the expression level of IFITM3.

By “GNA-12” and “IFITM3”, it is meant to refer, as the context will make clear, to the gene or RNA product or protein product.

In the present invention, expression level can be the RNA expression level of GNA-12 or IFITM3 in said biological sample.

Preferably, the method further comprises determining the RNA levels of GNA-12 or IFITM3 in the sample. The RNA levels of GNA-12 or IFITM3 may be determined by the use of at least two oligonucleotide primers, for example, the primers selected from Table 2.

More preferably, the RNA levels of GNA-12 or IFITM3 being determined in the present invention are GNA-12 or IFITM3 mRNA. It will be appreciated that detecting the presence of an increased level of GNA-12 or IFITM3 mRNA in a cell compared to the level present in a normal (non-cancerous) cell may aid in the assessment of a patient's risk of developing oral and nasopharyngeal cancers. Increased GNA-12 or IFITM3 mRNA in a sample compared to that found in a normal (non-cancerous) tissue sample may be indicative of oral or nasopharyngeal cancer. The increased levels may also suggest that the patient will benefit from a particular form of treatment, such as treatment with a cancer vaccine as herein disclosed.

The RNA levels of GNA-12 or IFITM3 may be determined by using specific oligonucleotide primers and a nucleic acid amplification technique such as the polymerase chain reaction (PCR). Oligonucleotide primers can be synthesised using methods well known in the art, for example using solid-phase phosphoramidite chemistry. Preferably, the oligonucleotide primers are at least 20 nudeotides in length, more preferably at least 25 nucleotides in length and still more preferably at least 29 nucleotides in length.

Suitable conditions for PCR amplification include amplification in a suitable 1×amplification buffer: 10×amplification buffer is 500 mM KCl; 100 mM Tris. Cl (pH 8.3 at room temperature); 15 mM MgCl2; 0.1% gelatin. single-stranded DNA primers, suitable for use in a polymerase chain reaction, are particularly preferred.

It will be appreciated that GNA-12 or IFITM3 mRNA may be identified by reverse-transcriptase polymerase chain reaction (RT-PCR) using methods well known in the art.

Primers which are suitable for use in a polymerase chain reaction (PCR; Saiki et al (1988) Science 239,487-491) are preferred. Suitable PCR primers may have the following properties: It is well known that the sequence at the 5′end of the oligonucleotide need not match the target sequence to be amplified.

It is usual that the PCR primers do not contain any complementary structures with each other longer than 2 bases, especially at their 3′ends, as this feature may promote the formation of an artifactual product called “primer dimer”. When the 3′ends of the two primers hybridize, they form a “primed template” complex, and primer extension results in a short duplex product called “primer dimer”.

Internal secondary structure should be avoided in primers. For symmetric PCR, a 40-60% G+C content is often recommended for both primers, with no long stretches of any one base. The classical melting temperature calculations used in conjunction with DNA probe hybridization studies often predict that a given primer should anneal at a specific temperature or that the 72° C. extension temperature will dissociate the primer/template hybrid prematurely. In practice, the hybrids are more effective in the PCR process than generally predicted by simple Tm calculations.

Optimum annealing temperatures may be determined empirically and may be higher than predicted. Taq DNA polymerase does have activity in the 37-55° C. region, so primer extension will occur during the annealing step and the hybrid will be stabilized. The concentrations of the primers are equal in conventional (symmetric) PCR and, typically, within 0.1- to 1-range.

Any of the nucleic acid amplification protocols can be used in the method of the invention including the polymerase chain reaction, QB replicase and ligase chain reaction. Also, NASBA (nucleic acid sequence based amplification), also called 3SR, can be used as described in Compton (1991) Nature 350,91-92 and AIDS (1993), Vol 7 (Suppl 2), S108 or SDA (strand displacement amplification) can be used as described in Walker et al (1992) Nucl. Acids Res. 20,1691-1696. The polymerase chain reaction is particularly preferred because of its simplicity.

When a pair of suitable nucleic acids of the invention is used in a PCR it is convenient to detect the product by gel electrophoresis and ethidium bromide staining. As an alternative to detecting the product of DNA amplification using agarose gel electrophoresis and ethidium bromide staining of the DNA, it is convenient to use a labelled oligonucleotide capable of hybridising to the amplified DNA as a probe. When the amplification is by a PCR the oligonucleotide probe hybridises to the interprimer sequence as defined by the two primers. The oligonucleotide probe is preferably between 10 and 50 nucleotides long, more preferably between 15 and 30 nucleotides long. The probe may be labelled with a radionuclide such as ³²P, ³³P and ³⁵S using standard techniques, or may be labelled with a fluorescent dye. When the oligonucleotide probe is fluorescently labelled, the amplified DNA product may be detected in solution (see for example Balaguer et al (1991) “Quantification of DNA sequences obtained by polymerase chain reaction using a bioluminescence adsorbent” Anal. Biochem. 195,105-110 and DiCesare et al (1993) “A high-sensitivity electrochemiluminescence-based detection system for automated PCR product quantitation” BioTechniques 15,152-157.

Amplification products can also be detected using a probe which may have a fluorophore-quencher pair or may be attached to a solid support or may have a biotin tag or they may be detected using a combination of a capture probe and a detector probe.

Fluorophore-quencher pairs are particularly suited to quantitative measurements of PCR reactions (eg RT-PCR). Fluorescence polarisation using a suitable probe may also be used to detect PCR products.

Other methods of detecting mRNA levels are included.

Methods for determining the relative amount of GNA-12 or IFITM3 mRNA include: in situ hybridisation (In Situ Hybridization Protocols. Methods in Molecular Biology Volume 33. Edited by K H A Choo. 1994, Humana Press Inc (Totowa, N.J., USA) pp 480p and In Situ Hybridization: A Practical Approach. Edited by D G Wilkinson. 1992, Oxford University Press, Oxford, pp 163), in situ amplification, northerns, nuclease protection, probe arrays, and amplification based systems; The mRNA may be amplified prior to or during detection and quantitation. ‘Real time’ amplification methods wherein the product is measured for each amplification cycle may be particularly useful (eg Real time PCR Hid et al (1996) Genome Research 6,986-994, Gibson et al (1996) Genome Research 6,995-1001; Real time NASBA Oehlenschlager et al (1996 Nov. 12) PNAS (USA) 93 (23), 12811-6. Primers should be designed to preferentially amplify from an mRNA template rather than from the DNA, or be designed to create a product where the mRNA or DNA template origin can be distinguished by size or by probing. NASBA may be particularly useful as the process can be arranged such that only RNA is recognised as an initial substrate.

Detecting mRNA includes detecting mRNA in any context, or detecting that there are cells present which contain mRNA (for example, by in situ hybridisation, or in samples obtained from lysed cells). It is useful to detect the presence of mRNA or that certain cells are present (either generally or in a specific location) which can be detected by virtue of their expression of GNA-12 or IFITM3 mRNA. As noted, the presence versus absence of GNA-12 or IFITM3 mRNA may be a useful marker, or low levels versus high levels of GNA-12 or IFITM3 mRNA may be a useful marker, or specific quantified levels may be associated with a specific disease state. It will be appreciated that similar possibilities exist in relation to using the GNA-12 or IFITM3 polypeptide as a marker.

In the present invention, expression level can also be the protein expression level of GNA-12 or IFITM3 in said biological sample.

Alternatively, the method further comprises determining the protein levels of GNA-12 or IFITM3 in the sample.

The methods of the invention also include the measurement and detection of the GNA-12 or IFITM3 polypeptide in test samples and their comparison in a reference sample. It will be appreciated that detecting the presence of an increased level of GNA-12 or IFITM3 polypeptides in a cell compared to the level present in a reference sample, .e.g a normal (non-cancerous) cell may aid in the assesssment of a patient's risk of developing oral and nasopharyngeal cancers. Increased GNA-12 or IFITM3 polypeptides in a sample compared to that found in a normal (non-cancerous) tissue sample may be indicative of oral or nasopharyngeal cancer.

The sample containing RNA and/or protein derived from the patient is conveniently a sample of the tissue in which cancer is suspected or in which cancer may be or has been found. These methods may be used for any cancer, but they are particularly suitable in respect of oral or nasopharyngeal cancers. The sample may also be blood, serum or lymph nodes which may be particularly useful in determining whether a cancer has spread. Alternatively, the sample may be tissue sample obtained surgically from a patient. Preferably, the tissue is epithelial tissues.

The methods of the invention involving detection of the GNA-12 or IFITM3 polypeptide are particularly useful in relation to historical samples such as those containing paraffin-embedded sections of tumour samples.

The amount of the GNA-12 or IFITM3 polypeptide may be determined in any suitable way.

It is preferred if the amount of the GNA-12 or IFITM3 polypeptide is determined using a molecule which selectively binds to GNA-12 or IFITM3 polypeptide. Suitably, the molecule which selectively binds to GNA-12 or IFITM3 is an antibody. The antibody may also bind to a natural variant or fragment of GNA-12 or IFITM3 polypeptide.

By “variants” of the polypeptide we include insertions, deletions and substitutions, either conservative or non-conservative, where such changes do not substantially alter the activity of the said GNA-12 or IFITM3.

Variants and variations of the polynucleotide and polypeptide include natural variants, including allelic variants and naturally-occurring mutant forms.

By “fragment of GNA-12 of IFITM3”, we include any fragment which retains activity or which is useful in some other way, for example, for use in raising antibodies or in a binding assay.

The antibodies for use in the methods of the in invention may be monoclonal or polyclonal.

The protein levels of GNA-12 or IFITM3 may be determined using any suitable protein quantitation method. In particular, it is preferred if antibodies are used and that the amount of GNA-12 or IFITM3 is determined using methods which include quantititative western blotting, enzyme-linked immunosorbent assays (ELISA) or quantitative immunohistochemistry.

As noted above, an increased level of GNA-12 or IFITM3 in a sample compared with a known normal tissue reference sample is suggestive of a tumorigenic sample.

In a preferred embodiment of the invention, antibodies will immunoprecipitate GNA-12 or IFITM3 proteins from solution as well as react with GNA-12 or IFITM3 protein on western or immunoblots of polyacrylamide gels. In another preferred embodiment, antibodies will detect GNA-12 or IFITM3 proteins in paraffin or frozen tissue sections, using immunocytochemical techniques.

Preferred embodiments relating to methods for detecting GNA-12 or IFITM3 include enzyme linked immunosorbent assays (ELISA), radioimmunoassay (RIA), immunoradiometric assays (IRMA) and immunoenzymatic assays (IEMA), including sandwich assays using monoclonal and/or polyclonal antibodies.

Exemplary sandwich assays are described by David et al in U.S. Pat. Nos. 4,376,110 and 4,486,530, hereby incorporated by reference.

Methods for detection also include immuno-fluoresence. Automated and semi-automated image analysis systems may be of use. Several formats for quantitative immunoassays are known. Such systems may incorporate: more than one antibody which binds the antigen; labelled or unlabelled antigen (in addition to any contained in the sample); and a variety of detection systems including radioisotope, colourimetric, fluorimetric, chemiluminescent, and enhanced chemiluminescent; enzyme catalysis may or may not be involved. Immunoassays may be homogenous systems, where no separation of bound and unbound reagents takes place, or heterogeneous systems involving a separation step.

Such assays are commonly referred to as eg enzyme-linked luminescent immunoassays (ELLIA), fluorescence enzyme immunoassay (FEIA), fluorescence immunoassay (FIA), enzyme immunoassay (EIA), luminescent immunoassay (LIA), latex photometrix immunoassay (LPIA).

Methods of cultivating the biological sample (e.g. sample cells) and isolating proteins are well known in the art. Cells can be harvested and lysed and the presence of the protein in the supernatant can be detected using antibodies. Such antibodies are useful in cancer diagnosis. Suitably, the antibodies of the invention are detectably labelled, for example they may be labelled in such a way that they may be directly or indirectly detected. Conveniently, the antibodies may be labelled with a radioactive moiety or a coloured moiety or a fluorescent moiety, or they may be linked to an enzyme. Typically, the enzyme is one which can convert a non-coloured (or non-fluorescent) substrate to a coloured (or fluorescent) product. The antibody may be labelled by biotin (or streptavidin) and then detected indirectly using streptavidin (or biotin) which has been labelled with a radioactive moiety or a coloured moiety or a fluorescent moiety, or the like or they may be linked to an enzyme of the type described above.

Anti-“GNA-12” or anti-“IFITM3” antibodies or fragments or derivatives thereof such as humanised antibodies or ScFv fragments or dAbs or other fragments which retain antigen-binding specificity may be useful for imaging, such as imaging of tumours in the patient using, for example, radioimmunoscintigraphy. Conveniently, the antibodies or fragments or derivatives thereof are labelled with a moiety which allows detection.

Suitably, the label is a radioactive moiety and, preferably, it contains 99mTc, or other suitable isotopes of technetium, or suitable isotopes of yttrium, indium, iodine or the like, all of which are well known in the art.

Preferably, the antibody is a monoclonal antibody or fragment thereof.

Anti-GNA-12 of anti-IFITM3 antibodies or fragments or derivatives thereof may be used therapeutically. For example, unconjugated antibodies or fragments or derivatives thereof may be used to induce an anti-idiotype response.

Alternatively, antibodies or fragments or derivatives thereof may be conjugated to a moiety which is directly or indirectly cytotoxic. Directly cytotoxic agents include, for example, radioisotopes and toxins such as ricin; indirectly cytotoxic agents include, for example, enzymes which can convert a relatively non-toxic prodrug into a cytotoxic drug. It is particularly preferred if peptides are made, based on the amino acid sequence of GNA-12 or IFITM3 (as shown in FIGS. 5 and 6), which allow for specific antibodies to be made.

Preferably, the reference is normal tissue. The reference, to which the determined expression levels the genes is compared, may be normal (healthy) tissue, such as normal epithelial cells, or any other reference tissue. The reference tissue can be non-cancerous tissue composed of normal tissue levels. A person skilled in the art will be able to determine, based on the appearance and histology of the tissue sample, whether the sample tissue is normal (healthy) or cancerous. The reference tissue can either originate from the subject the biological sample is collected from or from any other adequate source, for example, a subject not suffering from oral or nasopharyngeal cancers. Alternatively, cell lines, e.g human oral cancer cell lines, may be used to exogenously express the GNA-12 and IFITM3.

Preferably, the cancer is selected from the group consisting of oral and nasopharyngeal cancers.

Preferably, the biological sample is selected from the group consising of oral mucosal tissue, nasopharyngeal swab and mouth washing.

In accordance with another aspect of the invention, there is provided a method of detecting oral and nasopharyngeal cancers in a patient, the method comprising administering to the patient an anti-GNA-12 or anti-IFITM3 antibody or a fragment or derivative thereof labelled with a detectable label, allowing the labelled antibody to locate to the cancer, and imaging the cancer.

A further aspect of the invention provides the use of a nucleic acid which selectively hybridises to GNA-12 or IFITM3 mRNA in the manufacture of a reagent for diagnosing oral and nasopharyngeal cancers. Yet a further aspect of the invention provides the use of a nucleic acid which selectively hybridises to GNA-12 or IFITM3 mRNA in a method of diagnosing oral and nasopharyngeal cancers.

A further aspect of the invention provides the use of a molecule which selectively binds to GNA-12 of IFITM3 polypeptide or a natural fragment or variant thereof in the manufacture of a reagent for diagnosing or imaging oral and nasopharyngeal cancers.

In another aspect of the invention, there is provided a method of treating oral and nasopharyngeal cancers, the method comprising administering to the patient an effective amount of GNA-12 or IFITM3 polypeptide or a variant or fusion or fragment thereof, or an effective amount of a nucleic acid encoding a GNA-12 or IFITM3 polypeptide or a variant or fragment or fusion thereof, wherein the amount of said polypeptide or amount of said nucleic acid is effective to provoke an anti-cancer cell immune response in said patient.

The peptide or peptide-encoding nucleic acid constitutes a tumour or cancer vaccine. It may be administered directly into the patient, into the affected organ or systemically, or applied ex vivo to cells derived from the patient or a human cell line which are subsequently administered to the patient, or used in vitro to select a subpopulation from immune cells derived from the patient, which are then re-administered to the patient. If the nucleic acid is administered to cells in vitro, it may be useful for the cells to be transfected so as to co-express immune-stimulating cytokines, such as interleukin-2. The GNA-12 or IFITM3 polypeptide or peptide fragment may be substantially pure, or combined with an immune-stimulating adjuvant such as Detox, or used in combination with immune-stimulatory cytokines, or be administered with a suitable delivery system, for example liposomes.

The GNA-12 or IFITM3 polypeptide or peptide fragment may also be conjugated to a suitable cancer such as keyhole limpet haemocyanin (KLH) or mannan (see WO 95/18145 and Longenecker et al (1993) Ann. NYAcad. Sci. 690, 276-291). The peptide may also be tagged, or be a fusion protein. The nucleic acid may be substantially pure, or contained in a suitable vector or delivery system. Suitable vectors and delivery systems include viral, such as systems based on adenovirus, vaccinia virus, retroviruses, herpes virus, adeno-associated virus or hybrids containing elements of more than one virus. Non-viral delivery systems include cationic lipids and cationic polymers as are well known in the art of DNA delivery. Physical delivery, such as via a “gene-gun” may also be used. The peptide or peptide encoded by the nucleic acid may be a fusion protein, for example with β2-microglobulin.

The peptide fragment for use in a oral and nasopharyngeal cancer vaccine may be any suitable length fragment of the GNA-12 or IFITM3 polypeptide. In particular, it may be a suitable 9-mer peptide or a suitable 7-mer or 8-mer peptide. Longer peptides may also be suitable, but 9-mer peptides are preferred. Multiple epitopes, derived from the GNA-12 or IFITM3 polypeptide, may also be used. The term peptide includes a peptidomimetic. It also includes glycopeptides.

Suitably, any nucleic acid or peptide administered to the patient is sterile and pyrogen free. Naked DNA may be given intramuscularly or intradermally or subcutaneously. The peptides may be given intramuscularly, intradermally or subcutaneously.

It is particularly useful if the oral and nasopharyngeal cancer vaccine is administered in a manner which produces a cellular immune response, resulting in cytoxic tumour cell killing by NK cells or cytotoxic T cells (CTLs). Strategies of administration which activate T helper cells are particularly useful. It may also be useful to stimulate a humoral response. It may be useful to co-administer certain cytokines to promote such a response, for example interleukin-2, interleukin-12, interleukin-6, or interleukin-10. In addition, it may be useful to combine vaccination with strategies which increase MHC presentation on the surface of tumour cells, for example by co-administration of interferon-gamma or retinoic as is described in Nouri et al (1992) Eur. J. Cancer 28A, 1110-1115 and Seliger et al (1997) Scand. J. Immunol. 46,625-632. It may also be desirable to make modifications to the antigen (GNA-12 of IFITM3 polypeptide or part thereof) to enhance its presentation to the immune system.

It may also be useful to target the vaccine to specific cell populations, for example antigen presenting cells, either by the site of injection, use of targeting vectors and delivery systems, or selective purification of such a cell population from the patient and ex vivo administration of the peptide or nucleic acid (for example dendritic cells may be sorted as described in Zhou et al (1995) Blood 86, 3295-3301; Roth et al (1996) Scand. J. Immunology 43,646-651). For example, targeting vectors may comprise a tissue-or tumour-specific promoter which directs expression of the antigen at a suitable place.

Patients to whom the therapy is to be given, may have their tumours typed for overexpression or abnormal expression of GNA-12 of IFITM3.

A further aspect of the invention provides the use of an effective amount of GNA-12 or IFITM3 polypeptide or a variant or fusion or fragment thereof, or an effective amount of a nucleic acid encoding a GNA-12 or IFITM3 polypeptide or a variant or fragment or fusion thereof, in the manufacture of a medicament for treating oral and nasopharyngeal cancers.

In any aspect of the invention, there is provided a oral and nasopharyngeal cancer vaccine comprising a GNA-12 or IFITM3 polypeptide or variant or fragment thereof, or a nucleic acid encoding GNA-12 or IFITM3 polypeptide or fragment or variant thereof. It is known that inoculation with a nucleic acid vaccine, such as a DNA vaccine, encoding a polypeptide leads to a T cell response. In particular, MHC class I and class II-mediated interactions can be elicited.

Peptide products derived by cytosolic degradation of fragments of tumour-specific proteins, expressed de novo, are believed to gain access to the presentational pathways, mimicking the presentation of, for example, viral proteins, in infected cells. Presentation as neo-antigens or surrogate antigens in this novel context is believed to be a means of breaking immunological tolerance, and may lead to the generation of a tumour-specific immune response.

Conveniently, the nucleic acid vaccine may comprise any suitable nucleic acid delivery means. The nucleic acid, preferably DNA, may be naked (i.e. with substantially no other components to be administered) or it may be delivered in a liposome or as part of a viral vector delivery system.

It is believed that uptake of the nucleic acid and expression of the encoded polypeptide by dendritic cells may be the mechanism of priming of the immune response.

It is preferred if the vaccine, such as DNA vaccine, is administered into the muscle. It is also preferred if the vaccine is administered onto the skin.

It is preferred if the nucleic acid vaccine is administered with an adjuvant such as BCG or alum. Other suitable adjuvants include Aquila's QS21 stimulon (Aquila Biotech, Worcester, Mass., USA) which is derived from saponin, mycobacterial extracts and synthetic bacterial cell wall mimics, and proprietory adjuvants such as Ribi's Detox. Quil A, another saponin-derived adjuvant, may also be used (Superfos, Denmark).

Other adjuvants such as Freund's may also be useful. It may also be useful to give the GNA-12 or IFITM3 antigen conjugated to keyhole limpet haemocyanin, preferably also with an adjuvant.

Polynucleotide-mediated immunisation therapy of cancer is described in Conry et al (1996) Seminars in Oncology 23,135-147; Condon et al (1996) Nature Medicine 2,1122-1127; Gong et al (1997) Nature Medicine 3,558-561; Zhai et al (1996) J. Immunol. 156,700-710; Graham et al (1996) Int J. Cancer 65, 664-670; and Burchell et al (1996) pp 309-313 In: Breast Cancer, Advances in biology and therapeutics, Calvo et al (eds), John Libbey Eurotext, all of which are incorporated herein by reference.

The GNA-12 or IFITM3 polypeptide is an appropriate target for a cell-mediated response to oral and nasopharyngeal cancer or tumour cells which express the GNA-12 or IFITM3 polypeptide.

Therapeutic response to oral and nasopharyngeal cancer vaccine may usefully be monitored. Suitably, GNA-12 or IFITM3 specific antibody and CTL responses are monitored using methods well known in the art to assess the efficacy of the therapeutic response. Lymphoblastic transformation assays, lymphokine release assays, CTL response assays and serologic assays may be used. Detection of antigen-specific T lymphocytes by fluorescent-activated cell sorting (FACS) may also be used and is described in Altman et al (1996) Science 274, 94-96 and in WO 96/26962.

Preferably, the nucleic acid of the invention may be detectably labelled. For example, they may be labelled in such a way that they may be directly or indirectly detected. Conveniently, the nucleic acid is labelled with a radioactive moiety or a coloured moiety or a fluorescent moiety or some other suitable detectable moiety such as digoxygenin and luminescent or chemiluminescent moieties. The polynucleotides may be linked to an enzyme, or they may be linked to biotin (or streptavidin) and detected in a similar way as described for antibodies of the invention. Also preferably the nucleic acid of the invention may be bound to a solid support (including arrays, beads, magnetic beads, sample containers and the like).

The nucleic acid of the invention may also incorporate a “tag” nucleotide sequence which tag sequence can subsequently be recognised by a further nucleic acid probe. Suitable labels or tags may also be used for the selective capture of the hybridised (or non-hybridised) polynucleotide using methods well known in the art.

In another aspect of the invention, there is provided a method of treating oral and nasopharyngeal cancers in a patient, the method comprising administering to the patient a molecule that modulates the activity of the GNA-12 or IFITM3 gene or their products.

By “modulates the activity of the GNA-12 or IFITM3 gene or their products”, it is meant to refer to the activation, inhibition, delay, repression or interference of one or more of; the activity of GNA-12 or IFITM3; the RNA splicing or posttranslational processing to GNA-12 or IFITM3; the phosphorylation of GNA-12 or IFITM3; the level of expression of GNA-12 or IFITM3 including both mRNA expression and protein expression; or the sub-cellular localisation of GNA-12 or IFITM3. Preferably, the term “modulates” refers to inhibition of the level of expression of GNA-12 or IFITM3.

By “treating cancer” or “combating cancer”, it is meant to include treating, preventing or ameliorating the symptoms of oral and nasopharyngeal cancers and also includes preventing the recurrence of oral and nasopharyngeal cancers.

Preferably, the molecule that modulates the activity of the GNA-12 or IFITM3 gene or their products is a GNA-12 or IFITM3 antisense agent, siRNA or antibody. Antisense inhibition of human G-alpha-12 expression is described by Cowsert in U.S. Pat. No. 5,998,206, hereby incorporated by reference.

By “antisense agent”, it is meant to include agents which bind to GNA-12 or IFITM3 mRNA and, preferably, inhibit its translation; also included are agents which bind to the GNA-12 or IFITM3 gene and inhibit its transcription. Antisense agents can be designed by reference to the GNA-12 and IFITM3 sequences. Preferably, the antisense agent is an oligonucleotide.

Oligonucleotides are subject to being degraded or inactivated by cellular endogenous nucleases. To counter this problem, it is possible to use modified oligonucleotides, eg having altered internucleotide linkages, in which the naturally occurring phosphodiester linkages have been replaced with another linkage. For example, Agrawal et al (1988) Proc. Natl. Acad. Sci. USA 85, 7079-7083 showed increased inhibition in tissue culture of HIV-1 using oligonucleotide phosphoramidates and phosphorothioates. Sarin et al (1988) Proc. Natl. Acad. Sci. USA 85, 7448-7451 demonstrated increased inhibition of HIV-1 using oligonucleotide methylphosphonates. Agrawal et al (1989) Proc. Natl. Acad. Sci. USA 86, 7790-7794 showed inhibition of HIV-1 replication in both early-infected and chronically infected cell cultures, using nucleotide sequence-specific oligonucleotide phosphorothioates. Leither et al (1990) Proc. Natl. Acad. Sci. USA 87, 3430-3434 report inhibition in tissue culture of influenza virus replication by oligonucleotide phosphorothioates.

Oligonucleotides having artificial linkages have been shown to be resistant to degradation in vivo. For example, Shaw et al (1991) in Nucleic Acids Res. 19,747-750, report that otherwise unmodified oligonucleotides become more resistant to nucleases in vivo when they are blocked at the 3′end by certain capping structures and that uncapped oligonucleotide phosphorothioates are not degraded in vivo.

A detailed description of the H-phosphonate approach to synthesizing oligonucleoside phosphorothioates is provided in Agrawal and Tang (1990) Tetrahedron Letters 31, 7541-7544, the teachings of which are hereby incorporated herein by reference. Syntheses of oligonucleoside methylphosphonates, phosphorodithioates, phosphoramidates, phosphate esters, bridged phosphoramidates and bridge phosphorothioates are known in the art. See, for example, Agrawal and Goodchild (1987) Tetrahedron Letters 28,3539; Nielsen et al (1988) Tetrahedron Letters 29,2911; Jager et al (1988) Biochemistry 27,7237; Uznanski et al (1987) Tetrahedron Letters 28,3401; Bannwarth (1988) Hely. Chim. Acta. 71,1517; Crosstick and Vyle (1989) Tetrahedron Letters 30,4693; Agrawal et al (1990) Proc. Natl. Acad. Sci. USA 87,1401-1405, the teachings of which are incorporated herein by reference. Other methods for synthesis or production also are possible. In a preferred embodiment the oligonucleotide is a deoxyribonucleic acid (DNA), although ribonucleic acid (RNA) sequences may also be synthesized and applied.

The oligonucleotides useful in the invention preferably are designed to resist degradation by endogenous nucleolytic enzymes. In vivo degradation of oligonucleotides produces oligonucleotide breakdown products of reduced length. Such breakdown products are more likely to engage in non-specific hybridization and are less likely to be effective, relative to their full-length counterparts. Thus, it is desirable to use oligonucleotides that are resistant to degradation in the body and which are able to reach the targeted cells. The present oligonucleotides can be rendered more resistant to degradation in vivo by substituting one or more internal artificial internucleotide linkages for the native phosphodiester linkages, for example, by replacing phosphate with sulphur in the linkage. Examples of linkages that may be used include phosphorothioates, methylphosphonates, sulphone, sulphate, ketyl, phosphorodithioates, various phosphoramidates, phosphate esters, bridged phosphorothioates and bridged phosphoramidates. Such examples are illustrative, rather than limiting, since other intemucleotide linkages are known in the art. See, for example, Cohen, (1990) Trends in Biotechnology. The synthesis of oligonucleotides having one or more of these linkages substituted for the phosphodiester internucleotide linkages is well known in the art, including synthetic pathways for producing oligonucleotides having mixed internucleotide linkages.

Oligonucleotides can be made resistant to extension by endogenous enzymes by “capping” or incorporating similar groups on the 5′or 3 terminal nucleotides. A reagent for capping is commercially available as Amino- Link II from Applied BioSystems Inc, Foster City, Calif. Methods for capping are described, for example, by Shaw et al (1991) Nucleic Acids Res. 19,747-750 and Agrawal et al (1991) Proc. Natl. Acad. Sci. USA 88 (17), 7595-7599, the teachings of which are hereby incorporated herein by reference.

A further method of making oligonucleotides resistant to nuclease attack is for them to be “self-stabilised” as described by Tang et al (1993) Nucl. Acids Res. 21,2729-2735 incorporated herein by reference. Self-stabilized oligonucleotides have hairpin loop structures at their 3′ends, and show increased resistance to degradation by snake venom phosphodiesterase, DNA polymerase I and fetal bovine serum. The self-stabilized region of the oligonucleotide does not interfere in hybridization with complementary nucleic acids, and pharmacokinetic and stability studies in mice have shown increased in vivo persistence of self-stabilized oligonucleotides with respect to their linear counterparts.

In accordance with the invention, the inherent binding specificity of antisense oligonucleotides characteristic of base pairing is enhanced by limiting the availability of the antisense compound to its intend locus in vivo, permitting lower dosages to be used and minimizing systemic effects.

Thus, oligonucleotides are applied locally to achieve the desired effect. The concentration of the oligonucleotides at the desired locus is much higher than if the oligonucleotides were administered systemically, and the therapeutic effect can be achieved using a significantly lower total amount.

The local high concentration of oligonucleotides enhances penetration of the targeted cells and effectively blocks translation of the target nucleic acid sequences.

The oligonucleotides can be delivered to the locus by any means appropriate for localised administration of a drug. For example, a solution of the oligonucleotides can be injected directly to the site or can be delivered by infusion using an infusion pump. The oligonucleotides also can be incorporated into an implantable device which when placed at the desired site, permits the oligonucleotides to be released into the surrounding locus.

The oligonucleotides are most preferably administered via a hydrogel material. The hydrogel is noninflammatory and biodegradable. Many such materials now are known, including those made from natural and synthetic polymers. In a preferred embodiment, the method exploits a hydrogel which is liquid below body temperature but gels to form a shape-retaining semisolid hydrogel at or near body temperature. Preferred hydrogel are polymers of ethylene oxide-propylene oxide repeating units. The properties of the polymer are dependent on the molecular weight of the polymer and the relative percentage of polyethylene oxide and polypropylene oxide in the polymer. Preferred hydrogels contain from about 10 to about 80% by weight ethylene oxide and from about 20 to about 90% by weight propylene oxide. A particularly preferred hydrogel contains about 70% polyethylene oxide and 30% polypropylene oxide. Hydrogels which can be used are available, for example, from BASF Corp., Parsippany, N.J., under the tradename PluronicR.

In this embodiment, the hydrogel is cooled to a liquid state and the oligonucleotides are admixed into the liquid to a concentration of about 1 mg oligonucleotide per gram of hydrogel. The resulting mixture then is applied onto the surface to be treated, for example by spraying or painting during surgery or using a catheter or endoscopic procedures. As the polymer warms, it solidifies to form a gel, and the oligonucleotides diffuse out of the gel into the surrounding cells over a period of time defined by the exact composition of the gel.

The oligonucleotides can be administered by means of other implants that are commercially available or described in the scientific literature, including liposomes, microcapsules and implantable devices. For example, implants made of biodegradable materials such as polyanhydrides, polyorthoesters, polylactic acid and polyglycolic acid and copolymers thereof, collagen, and protein polymers, or non-biodegradable materials such as ethylenevinyl acetate (EVAc), polyvinyl acetate, ethylene vinyl alcohol, and derivatives thereof can be used to locally deliver the oligonucleotides. The oligonucleotides can be incorporated into the material as it is polymerized or solidified, using melt or solvent evaporation techniques, or mechanically mixed with the material. In one embodiment, the oligonucleotides are mixed into or applied onto coatings for implantable devices such as dextran coated silica beads, stents, or catheters.

The dose of oligonucleotides is dependent on the size of the oligonucleotides and the purpose for which is it administered. In general, the range is calculated based on the surface area of tissue to be treated. The effective dose of oligonucleotide is somewhat dependent on the length and chemical composition of the oligonucleotide but is generally in the range of about 30 to 3000 ug per square centimetre of tissue surface area.

The oligonucleotides may be administered to the patient systemically for both therapeutic and prophylactic purposes. The oligonucleotides may be administered by any effective method, for example, parenterally (eg intravenously, subcutaneously, intramuscularly) or by oral, nasal or other means which permit the oligonucleotides to access and circulate in the patient's bloodstream. Oligonucleotides administered systemically preferably are given in addition to locally administered oligonucleotides, but also have utility in the absence of local administration. A dosage in the range of from about 0.1 to about 10 grams per administration to an adult human generally will be effective for this purpose.

It will be appreciated from the foregoing that the invention contemplates the use of a polynucleotide, or antibody, to detect a cell expressing GNA-12 or IFITM3.

A further aspect of the invention provides the use of a molecule that modulates the activity of the GNA-12 or IFITM3 gene or their products in the manufacture of a medicament for combating oral and nasopharyngeal cancers.

In accordance with another aspect of the invention, there is provided a method of combating oral and nasopharyngeal cancers in a patient, the method comprising administering to the patient an antibody directed to GNA12 or IFITM3.

It will be appreciated that, with the advancements in antibody technology, it may not be necessary to immunise an animal in order to produce an antibody. Synthetic systems, such as phage display libraries, may be used. The use of such systems is included in the methods of the invention.

Monoclonal antibodies which will bind to GNA-12 or IFITM3 antigens can be prepared. The antigen-binding portion may be a part of an antibody (for example a Fab fragment) or a synthetic antibody fragment (for example a single chain Fv fragment [ScFv]). Suitable monoclonal antibodies to selected antigens may be prepared by known techniques, for example those disclosed in “Monoclonal Antibodies: A manual of techniques”, H Zola (CRC Press, 1988) and in “Monoclonal Hybridoma Antibodies: Techniques and Applications”, J G R Hurrell (CRC Press, 1982).

Chimaeric antibodies are discussed by Neuberger et al (1988,8th International Biotechnology Symposium Part 2,792-799).

Suitably prepared non-human antibodies can be “humanised” in known ways, for example by inserting the CDR regions of mouse antibodies into the framework of human antibodies.

The variable heavy (V_{H}) and variable light (V_{L}) domains of the antibody are involved in antigen recognition, a fact first recognised by early protease digestion experiments. Further confirmation was found by “humanisation” of rodent antibodies. Variable domains of rodent origin may be fused to constant domains of human origin such that the resultant antibody retains the antigenic specificity of the rodent parented antibody (Morrison et al (1984) Proc. Natl. Acad. Sci. USA 81,6851-6855).

That antigenic specificity is conferred by variable domains and is independent of the constant domains is known from experiments involving the bacterial expression of antibody fragments, all containing one or more variable domains. These molecules include Fab-like molecules (Better et al (1988) Science 240,1041); Fv molecules (Skerra et al (1988) Science 240, 1038); single-chain Fv (ScFv) molecules where the V_{H} and V_{L} partner domains are linked via a flexible oligopeptide (Bird et al (1988) Science 242,423; Huston et al (1988) Proc. Natl. Acad. Sci. USA 85,5879) and single domain antibodies (dAbs) comprising isolated V domains (Ward et al (1989) Nature 341,544). A general review of the techniques involved in the synthesis of antibody fragments which retain their specific binding sites is to be found in Winter & Milstein (1991) Nature 349,293-299.

By “ScFv molecules” we mean molecules wherein the V_{H} and V_{L} partner domains are linked via a flexible oligopeptide.

Fab, Fv, ScFv and dAb antibody fragments can all be made and expressed in and secreted from, for example, E. coli, thus allowing the facile production of large amounts of the said fragments.

Whole antibodies, and F (ab′) 2 fragments are “bivalent”. By “bivalent” we mean that the said antibodies and F (ab′) 2 fragments have two antigen combining sites. In contrast, Fab, Fv, ScFv and dAb fragments are monovalent, having only one antigen combining sites.

The antibody may be labelled with a directly or indirectly cytotoxic agent. Various of these agents have previously been attached to antibodies and other target site-delivery agents, and may readily be made by the person skilled in the art. In particular, the cytotoxic agent is preferably directly or indirectly toxic to cells in neovasculature or cells which are in close proximity to and associated with neovasculature.

By “directly cytotoxic” we include the meaning that the agent is one which on its own is cytotoxic. By “indirectly cytotoxic” we include the meaning that the agent is one which, although is not itself cytotoxic, can induce cytotoxicity, for example by its action on a further molecule or by further action on it.

In one embodiment the cytotoxic agent is a cytotoxic chemotherapeutic agent. Cytotoxic chemotherapeutic agents are well known in the art.

Cytotoxic chemotherapeutic agents, such as anticancer agents, include: alkylating agents including nitrogen mustards such as mechlorethamine (HN₂), cyclophosphamide, ifosfamide, melphalan (L-sarcolysin) and chlorambucil; ethylenimines and methylmelamines such as hexamethylmelamine, thiotepa; alkyl sulphonates such as busulfan; nitrosoureas such as carmustine (BCNU), lomustine (CCNU), semustine (methyl-CCNU) and streptozocin (streptozotocin); and triazenes such as decarbazine (DTIC; dimethyltriazenoimidazole-carboxamide); Antimetabolites including folic acid analogues such as methotrexate (amethopterin); pyrimidine analogues such as fluorouracil (5-fluorouracil; 5-FU), floxuridine (fluorodeoxyuridine; FUdR) and cytarabine (cytosine arabinoside); and purine analogues and related inhibitors such as mercaptopurine (6-mercaptopurine; 6-MP), thioguanine (6-thioguanine; TG) and pentostatin (2 -deoxycoformycin). Natural Products including vinca alkaloids such as vinblastine (VLB) and vincristine; epipodophyllotoxins such as etoposide and teniposide; antibiotics such as dactinomycin (actinomycin daunorubicin (daunomycin; rubidomycin), doxorubicin, bleomycin, plicamycin (mithramycin) and mitomycin (mitomycin C); enzymes such as L-asparaginase; and biological response modifiers such as interferon alphenomes. Miscellaneous agents including platinum coordination complexes such as cisplatin (cis-DDP) and carboplatin; anthracenedione such as mitoxantrone and anthracycline; substituted urea such as hydroxyurea; methyl hydrazine derivative such as procarbazine (N-methylhydrazine, MIH); and adrenocortical suppressant such as mitotane (o,pN-DDD) and aminoglutethimide; taxol and analogues/derivatives; and hormone agonists/antagonists such as flutamide and tamoxifen.

An example of a cytotoxic agent attached to antibodies is carbodiimide conjugation (Bauminger & Wilchek (1980) Methods Enzymol. 70, 151-159; incorporated herein by reference) may be used to conjugate a variety of agents, including doxorubicin, to antibodies or peptides.

Carbodiimides comprise a group of compounds that have the general formula R—N═C═N—RN, where R and RN can be aliphatic or aromatic, and are used for synthesis of peptide bonds. The preparative procedure is simple, relatively fast, and is carried out under mild conditions. Carbodiimide compounds attack carboxylic groups to change them into reactive sites for free amino groups.

The water soluble carbodiimide, 1-ethyl-3-(3-dimethylaminopropyl) carbodiimide (EDC) is particularly useful for conjugating a functional agent to a binding agent and may be used to conjugate doxorubicin to tumor homing peptides. The conjugation of doxorubicin and a binding moiety requires the presence of an amino group, which is provided by doxorubicin, and a carboxyl group, which is provided by the binding moiety such as an antibody or peptide.

In addition to using carbodiimides for the direct formation of peptide bonds, EDC also can be used to prepare active esters such as N-hydroxysuccinimide (NHS) ester. The NHS ester, which binds only to amino groups, then can be used to induce the formation of an amide bond with the single amino group of the doxorubicin. The use of EDC and NHS in combination is commonly used for conjugation in order to increase yield of conjugate formation (Bauminger & Wilchek, supra, 1980).

Other methods for conjugating a functional agent to a binding agent also can be used. For example, sodium periodate oxidation followed by reductive alkylation of appropriate reactants can be used, as can glutaraldehyde crosslinking. However, it is recognised that, regardless of which method of producing a conjugate of the invention is selected, a determination must be made that the binding moiety maintains its targeting ability and that the functional moiety maintains its relevant function.

In a further embodiment of the invention, the cytotoxic moiety is a cytotoxic peptide or polypeptide moiety by which we include any moiety which leads to cell death. Cytotoxic peptide and polypeptide moieties are well known in the art and include, for example, ricin, abrin, Pseudomonas exotoxin, tissue factor and the like. Methods for linking them to antibodies are also known in the art. The use of ricin as a cytotoxic agent is described in Burrows & Thorpe (1993) Proc. Natl. Acad. Sci. USA 90, 8996-9000, incorporated herein by reference, and the use of tissue factor, which leads to localised blood clotting and infarction of a tumour, has been described by Ran et al (1998) Cancer Res. 58, 4646-4653 and Huang et a! (1997) Science 275, 547-550. Tsai et a! (1995) Dis. Colon Rectum 38, 1067-1074 describes the abrin A chain conjugated to a monoclonal antibody and is incorporated herein by reference. Other ribosome inactivating proteins are described as cytotoxic agents in WO 96/06641. Pseudomonas exotoxin may also be used as the cytotoxic polypeptide agent (see, for example, Aiello et al (1995) Proc. Natl. Acad. Sci. USA 92, 10457-10461; incorporated herein by reference).

Certain cytokines, such as TNFI and IL-2, may also be useful as cytotoxic agents.

Certain radioactive atoms may also be cytotoxic if delivered in sufficient doses. Thus, the cytotoxic agent may comprise a radioactive atom which, in use, delivers a sufficient quantity of radioactivity to the target site so as to be cytotoxic. Suitable radioactive atoms include phosphorus-32, iodine-125, iodine-131, indium-111, rhenium-186, rhenium-188 or yttrium-90, or any other isotope which emits enough energy to destroy neighbouring cells, organelles or nucleic acid. Preferably, the isotopes and density of radioactive atoms in the compound of the invention are such that a dose of more than 4000 cGy (preferably at least 6000, 8000 or 10000 cGy) is delivered to the target site and, preferably, to the cells at the target site and their organelles, particularly the nucleus.

The radioactive atom may be attached to the binding agent in known ways. For example EDTA or another chelating agent may be attached to the binding moiety and used to attach ¹¹¹ln or ⁹⁰Y. Tyrosine residues may be labelled with ¹²⁵I or ¹³¹I.

The cytotoxic agent may be a suitable indirectly cytotoxic polypeptide. In a particularly preferred embodiment, the indirectly cytotoxic polpeptide is a polypeptide which has enzymatic activity and can convert a relatively non-toxic prodrug into a cytotoxic drug. When the targeting moiety is an antibody this type of system is often referred to as ADEPT (Antibody-Directed Enzyme Prodrug Therapy). The system requires that the targeting moiety locates the enzymatic portion to the desired site in the body of the patient (ie the site expressing I5-integrin, such as new vascular tissue associated with a tumour) and after allowing time for the enzyme to localise at the site, administering a prodrug which is a substrate for the enzyme, the end product of the catalysis being a cytotoxic compound. The object of the approach is to maximise the concentration of drug at the desired site and to minimise the concentration of drug in normal tissues (see Senter, P. D. et al (1988) Anti-tumor effects of antibody-alkaline phosphatase conjugates in combination with etoposide phosphate Proc. Natl. Acad. Sci. USA 85, 4842-4846; Bagshawe (1987) Br. J. Cancer 56, 531-2; and Bagshawe, K. D. et al (1988) □A cytotoxic agent can be generated selectively at cancer sites□ Br. J. Cancer. 58, 700-703.)

The cytotoxic agent may be a radiosensitiser. Radiosensitisers include fluoropyrimidines, thymidine analogues, hydroxyurea, gemcitabine, fludarabine, nicotinamide, halogenated pyrimidines, 3-aminobenzamide, 3-aminobenzodiamide, etanixadole, pimonidazole and misonidazole (see, for example, McGinn et al (1996) J. Natl. Cancer Inst. 88, 1193-11203; Shewach & Lawrence (1996) Invest. New Drugs 14, 257-263; Horsman (1995) Acta Oncol. 34, 571-587; Shenoy & Singh (1992) Clin. Invest. 10, 533-551; Mitchell et al (1989) Int. J. Radiat. Biol. 56, 827-836; Iliakis & Kurtzman (1989) Int. J. Radiat. Oncol. Biol. Phys. 16, 1235-1241; Brown (1989) Int. J. Radiat. Oncol. Biol. Phys. 16, 987-993; Brown (1985) Cancer 55, 2222-2228).

Also, delivery of genes into cells can radiosensitise them, for example delivery of the p53 gene or cyclin D (Lang et al (1998) J. Neurosurg. 89, 125-132; Coco Martin et al (1999) Cancer Res. 59, 1134-1140).

A further aspect of the invention provides the use of an antibody directed to GNA-12 or IFITM3 in the manufacture of a medicament for the combating oral and nasopharyngeal cancer.

Further aspects of the invention provide polypeptides, antibodies and nucleic acids of the invention for use in medicine.

In accordance with another aspect of the invention, there is provided a kit for aiding assessment of a patient's risk of developing oral and nasopharyngeal cancers, or likely severity or likelihood of progression of oral and nasopharyngeal cancers, or aiding in selection of a cancer treatment regime for the patient, or aiding in assessment of a cancer treatment regime for treating oral and nasopharyngeal cancers, the kit comprising at least one reagent for determing the amount of the expression level of GNA-12, and a package insert containing instructions using the kit.

Preferably, the kit of the present invention further comprising a reagent for determining the amount of the expression level of IFITM3.

Preferably, the reagents for the detection are one or more oligonucleotide primers. More preferably, the one or more oligonucleotide primers comprise, consist essentially of or consist of any one of the nucleotide sequences.

Preferably, the kit is an array or chip.

With the advent of microarray technology, it is now possible to study gene expression of thousands of genes simultaneously, either to sub-classify cancers, to compare the genetic changes of diseases at different stages of progression or to identify genes that can be used as prognostic indicators (Golub et a!, 1999; van't Veer et al, 2002).

In the present invention, the gene expression profiles of oral cancers are determined and genes that may be important in driving oral carcinogenesis are identified. Significantly altered gene expression patterns differ between oral cancers with differing aetiological factors or social habits thereby demonstrating that oral cancer development is associated with the activation/inactivation of specific genes depending on the causative agents.

The invention will now be described with reference to the following none limiting figures and examples.

All references herein mentioned are hereby incorporated by reference.

### FIGURES

FIG. 1(a) shows a principle component analysis (PCA) of 22, 284 genes demonstrating that the global gene expression pattern of OSCC is distinct from normal oral mucosa.

FIG. 1(b) shows a principle component analysis (PCA) of 1068 genes demonstrating that the global gene expression patterns of OSCC from betel quid chewers are different from smokers.

FIG. 2 shows the quantitative PCR analysis of genes of interest. (a) Validation by qPCR analysis on 5 genes (MMP1, PLAU, MAGED-4, GNAl2 & IFITM3) and 1 gene (NMU) found to be up-regulated or down-regulated in the microarray analysis respectively when comparing tumour versus normal tissues. (b) Validation by qPCR analysis of 4 genes (TGF-a, CXCL-9, ITGB4 and KRT10) found to be differentially expressed between specimens from betel quid chewers (BT) and smokers (ST).

FIG. 3 shows an immunohistochemistry analysis of TGF-α (a-d) and ITGB4 (e-h). Consistent with the microarray results, tissue specimens from betel quid chewing patients showing lack of TGF-α expression (a, b) in comparison to tissues from patients who smoke (c, d). Similarly, differential expression of ITGB4 was seen between betel quid chewers (e, f) and smokers (g, h).

FIG. 4 shows the cDNA sequence of GNA-12.

FIG. 5 shows the amino acid sequence of GNA-12.

FIG. 6 shows the cDNA sequence of IFITM3.

FIG. 7 shows the amino acid sequence of IFITM3.

FIG. 8 shows the Tables referred to in this specification.

### EXAMPLE 1

Materials and Methods

Tissue Specimens and Collection Procedure

Oral squamous cell carcinoma (OSCC) and normal oral mucosal tissue samples were obtained from patients who were treated surgically. Seventeen specimens were included in this study: 5 OSCC tissues were paired with 5 normal oral mucosae from the same individuals who were BQ chewers; 4 OSCC tissues were paired with 2 normal oral mucosae from the same individuals who were smokers; 1 normal oral mucosa was also obtained from an unrelated individual with a smoking habit. Informed consent was obtained from all individuals and this project was approved as part of a nationwide study on ‘Oral Cancer and Precancer in Malaysia’ by the Medical Ethics Committee, Faculty of Dentistry, University of Malaya (Medical Ethics Clearance Number: DP OP0306/0018/L) and endorsed by the Ministry of Health, Malaysia. Tissue specimens in excess of diagnostic value were snap-frozen in liquid nitrogen and stored until use. The tumours represent a variety of sub-sites within the oral cavity and stages of disease (Table 1). Data on patient demographics, habits and disease stage were obtained from the Oral Cancer Research and Coordinating Centre (OCRCC), University of Malaya.

RNA Extraction

Tissue specimens were processed in a cryostat to avoid thawing of tissues. Initially, a reference slide was made to confirm the diagnosis and to gauge the percentage of epithelium or tumour cells. The tissue specimens were macrodissected to ensure that there was at least 70% tumour cells or for normal mucosa, at least 70% keratinocytes. RNA was extracted from a total of 100-200 micron sections depending on the size of the tissue and processed using the RNeasy Micro kit (Qiagen, USA) according to manufacturer's recommendations. The RNA quality and quantity was accessed using the Agilent 2100 Bioanalyzer and Nanodrop respectively. Only specimens with the RNA Integrity Number (RIN) of more than 7.5 were used for the microarray experiments.

Microarray Experiments

Twenty-five nanograms of total RNA was used in the two-cycle target labeling assay according to manufacturer's recommendations (Affymetrix; Santa Clara, USA). In brief, single-stranded and then double-stranded (ds) cDNA was synthesized from the poly (A)+ mRNA present in the isolated total RNA. This was followed by amplification of the cRNA, a second round of cDNA synthesis and then the synthesis of biotin-labeled cRNA. Twenty micrograms of the resulting biotin-labelled cRNA was fragmented and 15 μg was hybridized at 45 ° C. with rotation for 16 hours (Affymetrix GeneChip Hybridization Oven 640) to probes present on an Affymetrix HG-U133A array. The GeneChip arrays were washed and then stained (Streptavidin-phycoerythrin) in a GeneChip Fluidics Station 400 using the EukGE-WS2v4 protocol. Subsequently, the GeneChip was scanned on an Agilent GeneArray scanner.

Data Analysis

The intensities of the Affymetrix Arrays were measured by Microarray Suite 5.0 software (Affymetrix Santa Clara, USA). Intensity data were normalized to a target value (trimmed mean probe set signal of an array excluding lowest 2% and highest 2%) of 120. We excluded probe sets on the array that were assigned as ‘absent’ call in all samples. Following this, we looked at the global gene expression in all the samples by principal component analysis (PCA). A two tailed Student's t-test was used for comparison of average gene expression signal intensity between the tumours and normals to identify the differentially expressed transcripts. A p value of 0.001 was defined for statistical significance. Subsequently, fold ratios were calculated for gene transcripts that showed a statistically significant difference. Only gene transcripts that exhibited at least 2 fold change were included for further analysis. To identify genes that were differentially expressed between OSCC in BQ chewers and smokers, genes determined to be significantly changed between tumours and normals in the individual categories (2-fold at p<0.01 for chewers and at p<0.05 for smokers) were subjected together to principal component analysis (PCA). The squares of eigen vector coefficients of PC#1, PC#2 and PC#3 were used to select genes that were altered. Again, fold change ratios were calculated and only the genes that exhibited at least 1.8 fold change were included for further analysis.

Validation of Microarray Results

To validate the results of the microarray analysis, 10 representative genes thought to be of biological significance were subjected to realtime quantitative PCR (qPCR). In addition, expression of proteins of interest was observed using immunohistochemical techniques.

Quantitative Polymerase Chain Reaction (qPCR) cDNA was reverse transcribed from 50 ng total RNA from the same samples used in the microarray experiments using the Sensiscript Reverse Transcription Kit (Qiagen, USA). Real-time gPCR was performed using the ABI7000 DNA sequence analyzer and a SYBR green-based procedure. Oligonucleotide pairs for use as PCR primers were designed using Primer Express software (Applied Biosystems) using the reference sequences from which the microarray probes were generated. Primer sequences are shown in Table 2. Realtime qPCR reaction components were assembled in 96-well plates in 25 μl volumes, containing 10 μM each primer, 75 ng of cDNA in 1× concentration of SYBR green dye in triplicates.

Immunohistochemistry

Immunohistochemistry was performed using the Dakocytomation Envision®⁺Dual Link System-Peroxidase (DAB⁺) kit (Dako, USA), according to the manufacturer's specifications. Briefly, 5 μm sections of paraffin-embedded tissues from specimens included in the microarray analysis were deparaffinized with 2 xylene incubations at 5 minutes each and rehydrated in graded ethanol for 3 minutes each. The tissue sections were treated with 10 mM sodium citrate pH 6.0 for 10 minutes in a microwave for antigen retrieval. The sections were incubated with primary antibody (mouse monoclonal anti-integrin beta-4 (ITGB4) antibody (1:100; Chemicon, USA), rabbit polyclonal anti-TGF-α (1:75; Abcam, USA) followed by a peroxidase labeled polymer conjugated to goat anti-mouse/rabbit antibody for 30 minutes each at room temperature. The peroxidase reaction was developed using diaminobenzidine (DAB) as a chromogen. Negative controls, in which non-immune sera were used to replace the primary antibodies, were also performed.

Results

Global Gene Expression Pattern of OSCC are Different from Normal Oral Mucosa

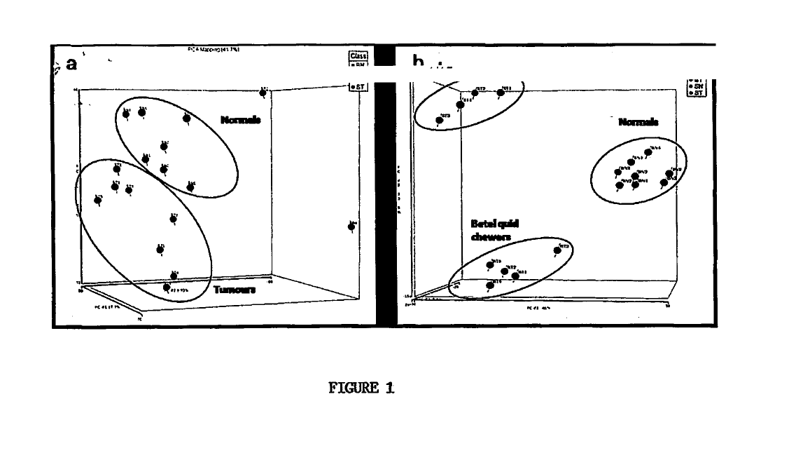

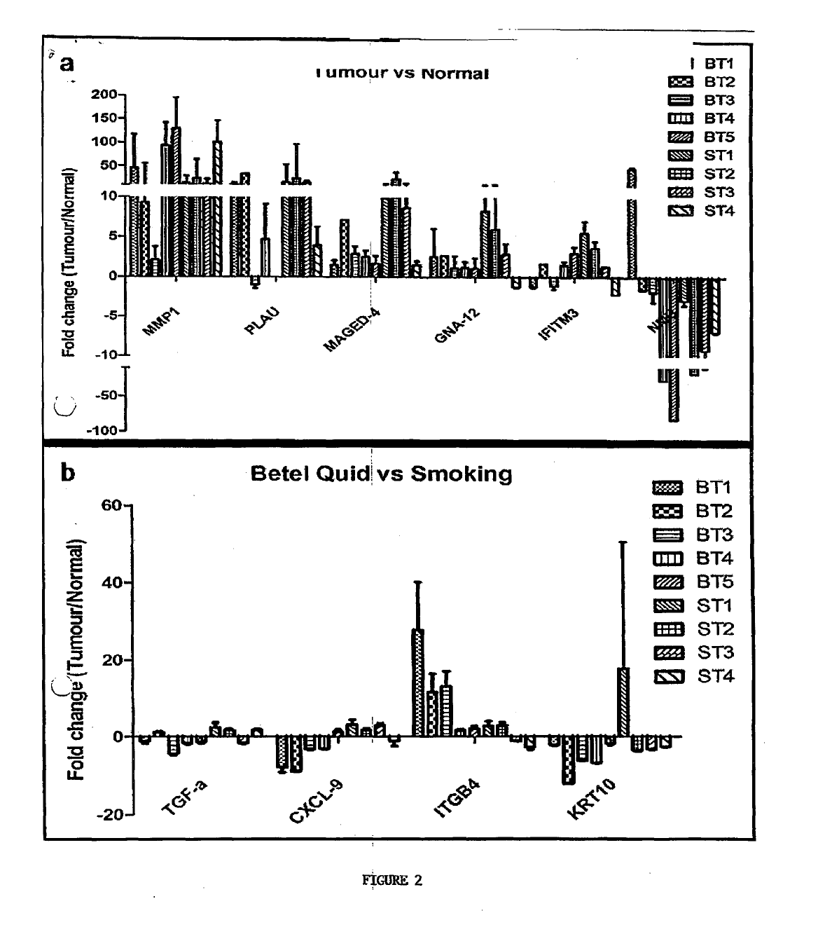

Tissue specimens were analyzed based on their global gene expression patterns using PCA. In this analysis, 8/9 tumour specimens grouped together distinctly from the normals suggesting that the tumour specimens have a unique pattern of gene expression by comparison to the normal tissues (FIG. 1a). Two hundred and eighty one genes (281) were found to be significantly changed (p<0.001) between normal and tumour tissues by at least 2 fold. Genes identified as showing up-regulation (172 genes; Table 3) included members of the matrix metalloproteinase (MMP) family such as MMP1, MMP3 and MMP10, signal transduction molecules such as GNA 12, and regulators of transcription such as MAGE-D4. By contrast, 109 genes were found to be down-regulated between tumours and normals (Table 4), including genes regulating apoptosis such as DAPK2; the immune response such as NCK1, epidermal differentiation such as EMP1 and EHF, cell adhesion molecules such as SEMA4D, enzymes involved in the metabolism and detoxification of carcinogens such as ALDH3A2 and CYP2E1 and molecules involved in signaling such as NMU (neuromedin). The expression of specific genes was confirmed by gPCR using the same input RNA that were used in the microarray experiments. Consistent with the microarray analysis, MMP1I, PLAU, MAGE-D4, GNAl2 and 1FITM3 were shown to be overexpressed whilst NMU mRNA expression was down-regulated (FIG. 2a). These data confirm that distinct gene expression profiles exist between tumour and normal tissues and suggest that these molecular changes may be the responsible for driving oral carcinogenesis.

Significant Expression Patterns of OSCC Associated with BQ Chewing are Distinct from those Associated with Smoking

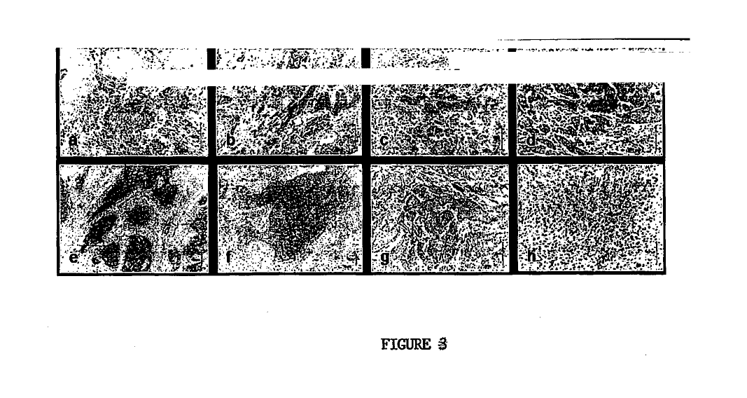

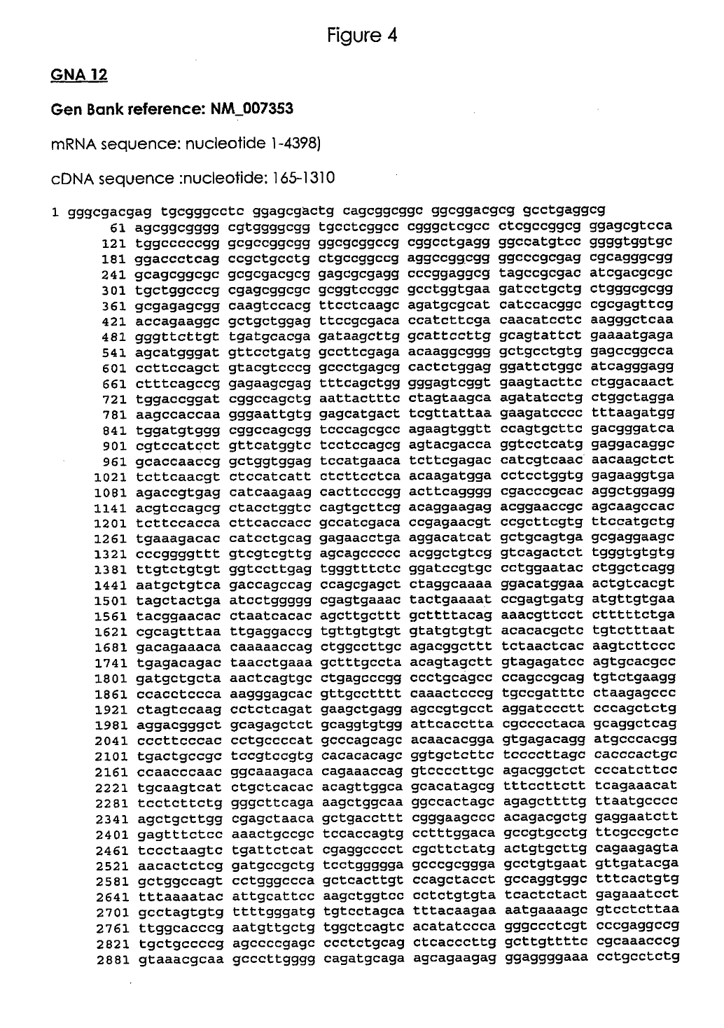

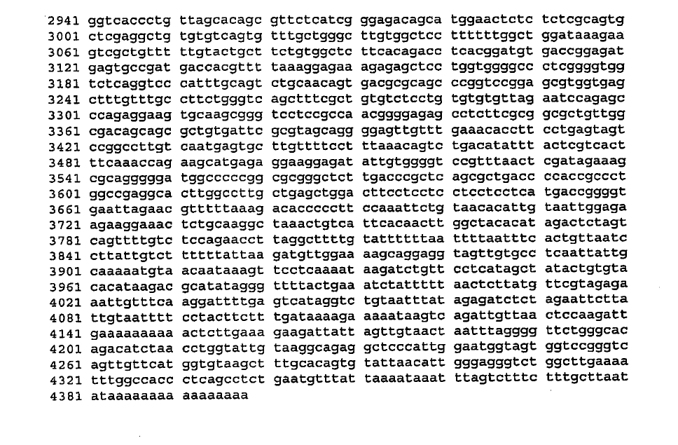

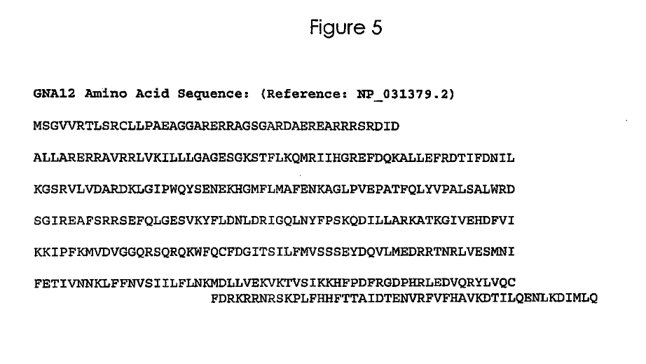

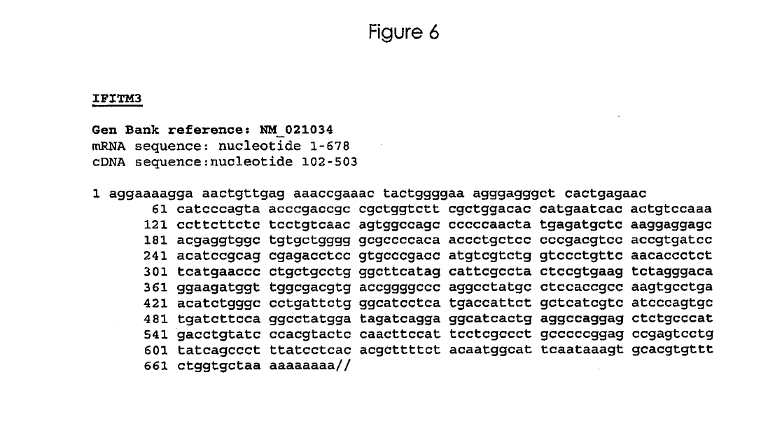

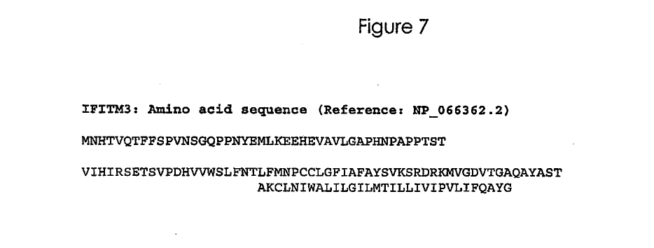

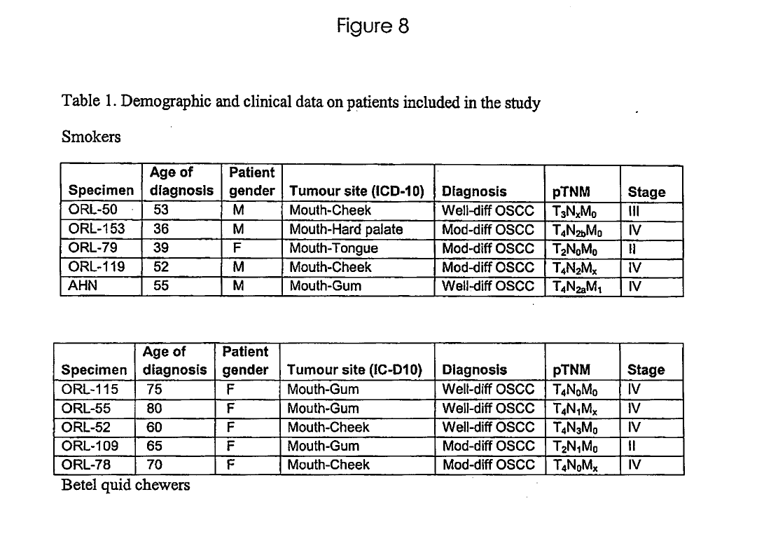

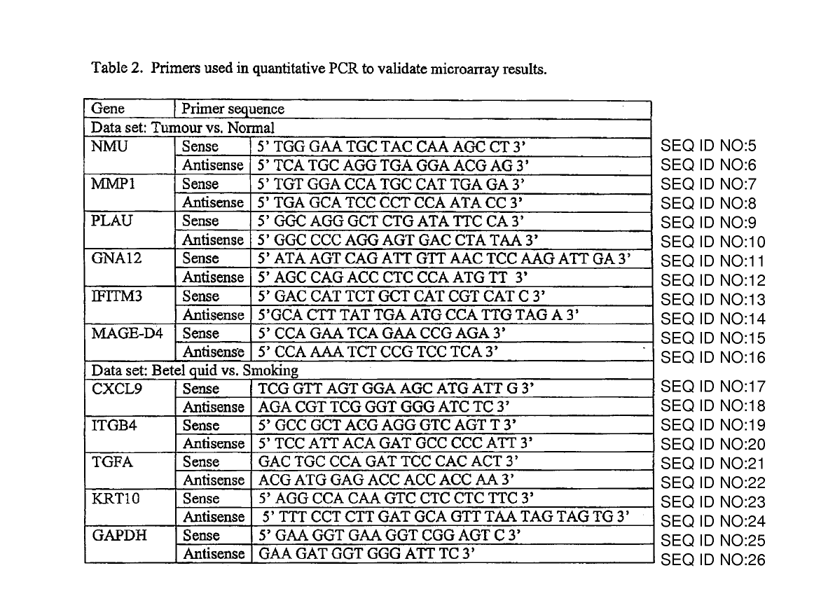

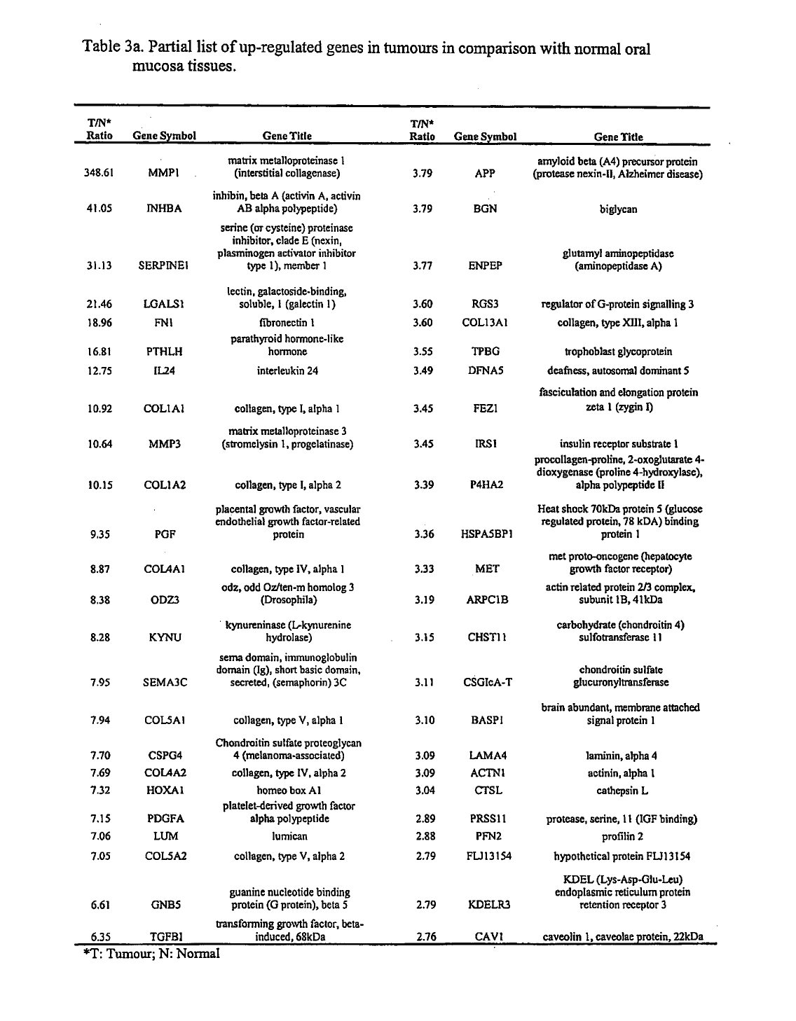

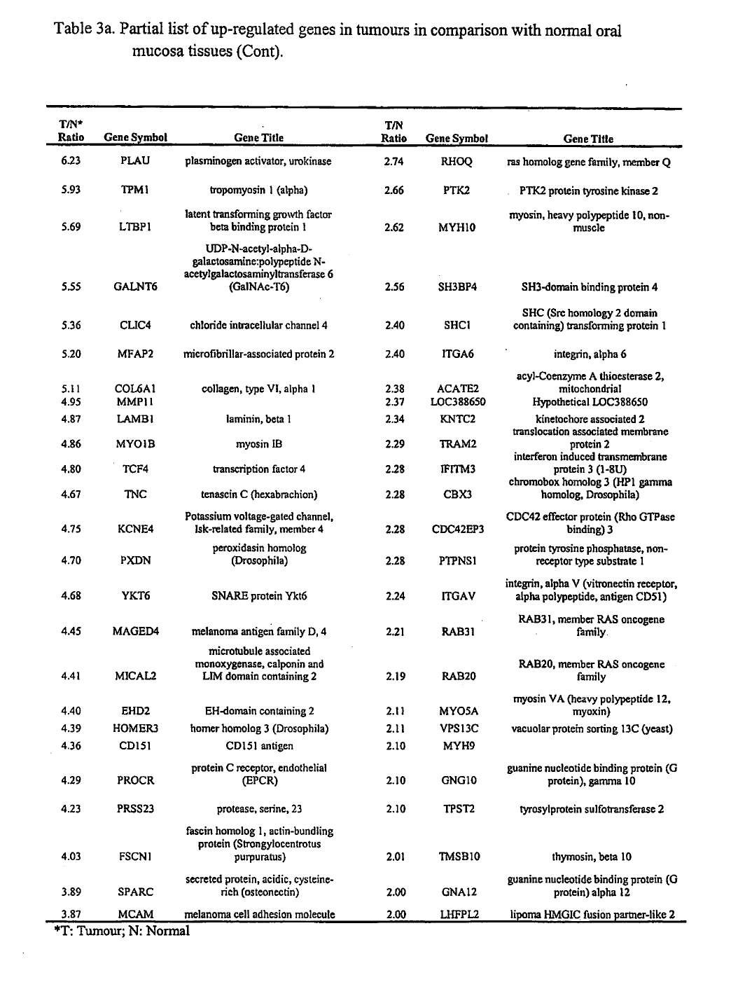

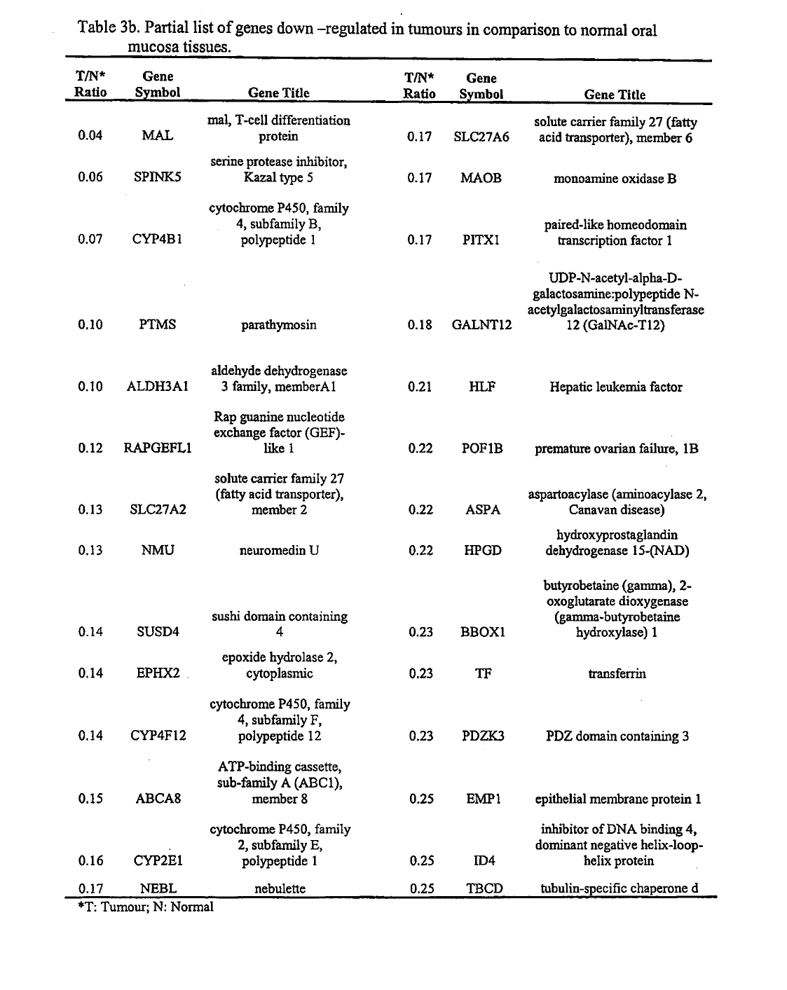

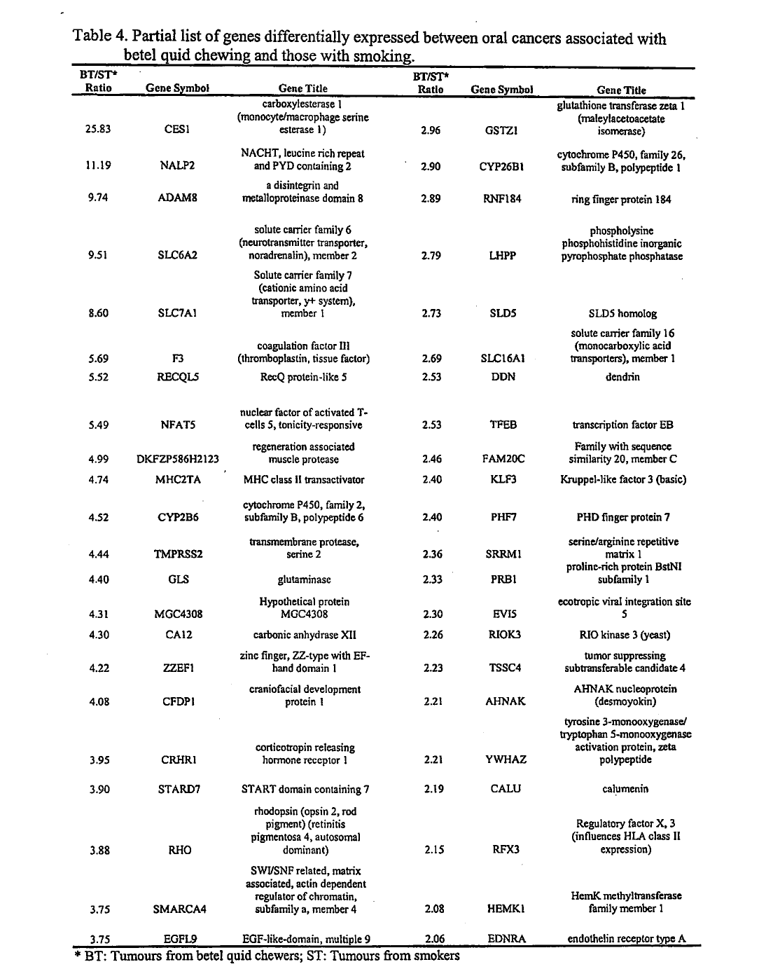

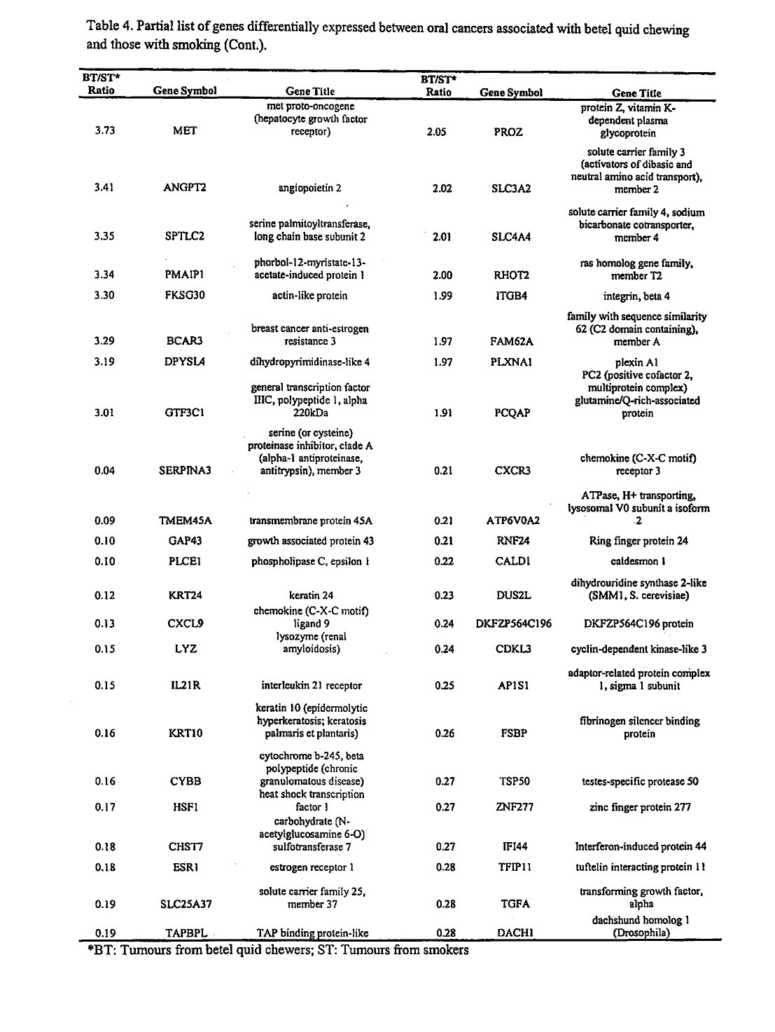

Genes differentially expressed between tumours associated with BQ chewing and those associated with smoking were identified from a total of 1053 genes found to be significantly altered either in the smoking or BQ chewing group. Data were transformed relative to respective mean normal values for comparison of the two cancers directly. PCA of these genes demonstrated that these 2 groups have distinct gene expression patterns and that PC#2 has a difference between these groups (FIG. 1b). Genes were selected if the squared eigen vector coefficient of PC#2 was greater than twice the squared value of PC#1 and PC#3. One hundred and eighty three genes were identified as differentially expressed by at least 1.8 fold between the 2 groups, and these were analysed further (Table 5). Sixty-seven genes were identified to be up-regulated in the BQ associated tumours in comparison to those associated with smoking and these included genes involved in signaling such as EDNRA and Met, cell adhesion molecules such as ITGB4, regulators of apoptosis such as PMAIP1, regulators of the cell cycle such as BCAR and metastasis-associated genes such as MMP24. One hundred and sixteen genes were found to be down-regulated in BQ associated OSCC by comparison to specimens from smokers and included genes involved in epithelial differentiation such as KRT10, cell signaling and growth such as TGF-α, cell adhesion molecules such as CX3CR1 and CX3CL1 and regulators of apoptosis such as DAPK2. Using qPCR, we confirmed the up-regulation of ITGB4 and the under expression of TGF-α, CXCL-9 and KRT-10 in samples from BQ chewers (FIG. 2b). The over-expression of ITGB4 and under-expression of TGF-α was also confirmed in all the BQ associated tissues tested using immunohistochemistry (FIG. 3).

Discussion

Some cancers have been associated with distinct geographical location and ethnic differences. This may be linked to exposure to distinct carcinogens, environmental factors, certain common practices within the population or due to genetic factors. In the case of oral cancers, oral habits such as BQ chewing and smoking have been long identified as the common causative factors of cancer onset. Although the aetiological roles of BQ and smoking are well-established, the precise contribution of these carcinogens to the genetic deregulation contributing to cancer formation remains unclear. It is likely that in genetically susceptible individuals, prolonged exposure to carcinogens in either BQ or tobacco smoke results in the activation of certain pathways which lead to malignant transformation. Indeed, this has been substantiated by the molecular analyses of genes and proteins associated with cancers. However, previous studies investigating the differences in genetic background of cancers associated with different aetiological factors have mostly focused on an individual gene, pathway or a small panel of interesting molecular candidates (Bradley et al., 2001; Lim et al., 2005; Thongsuksai et al., 2003). In the present example, a high-throughput whole genome microarray platform was used to determine the global gene expression changes in OSCC and demonstrated that the gene expression changes in BQ associated tumours are different to those caused by smoking.

Genes that are differentially expressed between tumour and normal tissues regardless of the habits of the patients have been identified. Several genes were selected based on their reported roles in carcinogenesis or their potential as prognostic or therapeutic targets. A common characteristic of the genes selected for further validation is that they confer a growth advantage either in promoting cellular proliferation, maintaining pluripotency and/or conferring cellular motility. One of these genes was MAGE-D4 (previously known as MAGE-1 or MAGE-E1), a member of the MAGE family of genes. MAGE proteins encode melanoma antigens and were originally discovered as antigens that are recognized by cytotoxic T lymphocytes (van der Bruggen et al, 1991). Overexpression of this protein has been described for glioblastomas and non-small cell lung cancer (Ito et al, 2006; Sasaki et al, 2001) and in the latter, its expression correlated with cell proliferation. Cell signaling is an integral part of cell survival and cancer growth. Many of these signal transmission occur via second messengers systems that are controlled by heterotrimeric G proteins and their linked receptors (Dorsam & Gutkind, 2007).

It was found in the present example that one of these G proteins, GNAl2 to be over-expressed in OSCC specimens, suggesting that it may have a role in driving oral cancer development. GNAl2 functions in transducing extracellular signals to activate Rho signaling (Tanabe at al, 2004) and has been reported to be highly expressed in breast and prostate cancer, where in vitro evidence suggest that it plays an important role in regulating cancer invasion and metastasis (Kelly at al, 2006a; Kelly at al, 2006b).

Another gene that was overexpressed in OSCC here was IFITM3, a member of the IFN-inducible transmembrane gene family. Members of this family are induced by activation of the beta-catenin signalling pathway and hence have been postulated to be partly responsible for the maintenance and propagation of the pluripotent state of cells. IFITM3 specifically, have been reported to be responsible for the specification of germ cell fate in mice (Saitou at al, 2003). Andreu and colleagues reported the up-regulation of IFITM3 in colorectal cancers of various stages and suggested that this expression maybe relevant for the clinical diagnosis of these cancers (Andreu et al, 2006). Its function in the maintenance of the pluripotent state of a cell and its overexpression in cancers including OSCC make IFITM3 an interesting target to study further in the context of oral carcinogenesis.

Genes that were down-regulated between tumours and normals were also examined in this study. One such gene was neuromedin (NMU), an observation that confirmed a previous report on the downregulation of NMU in oral cancers (Alevizos at al, 2001).

Consistently, the NMU promoter was observed to be hypermethylated in OSCC cell lines and tumour tissues (Tokumaru et al, 2004). Taken together, the data strongly support the view that NMU may have tumour suppressive properties in OSCC.

Different carcinogens give rise to different mutational profiles that can be detected in cancer cells. In the present example, OSCC from individuals who chew BQ to those who smoked were compared. BQ used by individuals in this study commonly contain areca nut, betel leaf, lime and tobacco (Ramanathan & Lakshimi, 1976). Although tobacco may be a common component between the 2 groups, the BQ chewers are exposed to an additional carcinogen through the constituents of the areca nut (IARC, 2003). Furthermore, smokeless tobacco has been shown to produce an array of carcinogenic substances that is quite distinct to those produced by smoking (Brunnemann & Hoffmann, 1992).

In order to look at effects of aetiological factors/social habits on gene expression patterns, the genes that were differentially expressed by 1.8-fold between cancers that are associated with BQ chewing with those that were from smokers were determined. For the first time, it was demonstrated here that gene expression patterns differ in oral cancers that are associated with BQ chewing to those associated with smoking, suggesting that the genetic progression of oral cancers associated with different risk factors are different. This is consistent with Ibrahim and colleagues who demonstrated that although a panel of genes may be common between 2 groups of patients with distinct risk factors, some of these genes are differentially expressed (Ibrahim at al, 2003).

Some genes that were found to be differentially expressed between BQ chewers and smokers in the present example are known to play a prominent role in signaling pathways. One particular gene is that encoding transforming growth factor-alpha (TGF-α). Self-sufficiency in growth signals is a hallmark of cancer (Hanahan & Weinberg, 2000) and many growth factors have been shown to stimulate oral keratinocytes proliferation including TGF-α. Up-regulation of TGF-α have been reported in many cancers including oral tumours, (Derynck et al, 1987; Grandis & Tweardy, 1993; Issing at al, 1993) and it is associated with poor survival (Issing et al., 1993). Interaction of TGF-α with the epidermal growth factor receptor (EGFR) leads to a cascade of intracellular signalling pathways to promote cell proliferation (van der Geer et al, 1994) and to facilitate cell invasion (McCawley et al, 1998). Interestingly, in the present example, overexpression of TGF-a is only seen in tumour samples from patients who smoke. Tobacco smoke has been shown to induce the expression of TGF-a (Moraitis et al, 2005; Richter et al, 2002) leading to EGFR dependent COX-2 activation (Moraitis et al., 2005). The activation of COX-2 has also been reported to drive cell proliferation by enhancing EGFR signaling via a positive feed-back loop hence amplifying the tumour promoting properties of tobacco smoke (Pai et al, 2002). This may partially explain our data and suggests that TGF-a may play a more important role in tobacco smoke related OSCC than in those provoked by BQ chewing.

Previous studies have shown that a robust immunological response in tumours is indicative of good prognosis. Chemokines are a group of superfamily of small cytokine-like molecules that regulate leukocyte trafficking but appear to have more complex roles in cancer. The interferon inducible chemokine CXCL-9 has been shown to be differentially expressed between BQ chewers and smokers such that the expression was down regulated in BQ chewers and up regulated in smokers. As the expression of chemokines could be modulated by cigarette smoke, this may explain the differences of CXCL-9 expression patterns in the 2 different groups of patients. Interestingly, Haque and colleagues have demonstrated that upon stimulation of cytokine production, IFN-y levels in cells from patients with oral submucous fibrosis (OSF) were significantly lower than controls (Haque et al, 2000) suggesting that individuals who were chronically exposed to BQ may have an impairment in the production of this cytokine and by extension would be unable to induce chemokines such as CXCL-9. CXCL-9 amongst other chemokines is capable of activating the phosphoinositide 3-kinase (PI3K) and mitogen-activated protein kinase (MAPK) pathways via its receptor (CXCR3) to elicit actin remodelling which is important for cell motility (Kouroumalis et al, 2005). This activation may be achieved via G coupled protein receptors such as G12/13 that is also seen to be overexpressed in our OSCC samples. Notably, in the present example, CXCR3 is also up-regulated in oral cancers associated with smoking. Interferon-based targeted therapies have been introduced for the treatment of cancers and chemokines such as CXCL-9 are directly induced by such therapies (Hutson et al, 2003) therefore, it is pertinent to understand the role CXCL-9/CXCR3 to ascertain the scenario where the expression of these molecules could promote or repress tumour formation to ensure maximum benefit of such therapies.

In the present example, it was found that integrin β4 (β4) was over-expressed in OSCC in the BQ chewers compared to the smokers where it was overexpressed in the former. β4 heterodimerizes with integrin α6 and together they form the receptor for laminin (Kikkawa et al, 2000; Mercurio, 1995). α6β4 have been reported to play a role in both the initiation of tumour growth by promoting anchorage independent growth (Bertotti et al, 2006) and in facilitating cellular invasion by stimulating cell motility (Chung at al, 2004; Mercurio et al, 2001). Therefore it is not surprising that overexpression of 134 have been demonstrated in different tumours including head and neck (Carey et al, 1987; Carico et al, 1993; Lotz et al, 1990; Mariani Costantini et al, 1990). Furthermore, (34 has also been reported to cooperate with Met to enhance its transforming properties in vitro and in vivo (Bertotti et al, 2005). Notably, Met was also seen to be overexpressed in this study particularly in specimens associated with BQ chewing and whether or not both Met and a664 collaborate in driving oral carcinogenesis in these patients is currently unknown. It is also worth noting that expression of a6134 in differentiated cells is able to modulate the growth of basal cells and contribute to carcinogenesis (Owens at al, 2003; Watt, 2002). Consistent with previous studies indicating that 64 expression is observed in suprabasal keratinocytes, we show that this pattern of expression is also seen in OSCC associated with BQ chewing. Chen and colleagues have demonstrated that BQ extract is able to induce cell motility through the activation of the Src family kinases (Chen, unpublished). Fyn, one of the Src kinases has been shown to phosphorylate 64 to facilitate cell motility in keratinocytes (Mariotti et al, 2001). Although cell motility can be induced by several factors and facilitated by different pathways, the ability of BQ extract in inducing Fyn and the simultaneous overexpression of Met makes it tempting to hypothesize that cell signalling via the integrin pathway may be particularly important in OSCC in patients who chew BQ. A differential expression of β4 was also reported previously in a study looking at oral cancers in 2 distinct populations (Ibrahim at al., 2003).

Keratins originally presumed to be merely structural proteins have recently been shown to be able to modulate cell signaling. Recent evidence in in vitro and in vivo models suggest that expression of KRT10, a keratin normally expressed in postmitotic suprabasal cells of the mucosa directly induces cell cycle arrest and affects cell proliferation by inhibiting RB1 phosphorylation, cyclin D1 expression, interaction and impairment of Akt and PKCξ (Paramio et al, 1999; Santos et al, 2002). Further, tumour formation was reported to be delayed in KRT10 transgenic mice in comparison to non-transgenic animals and KRT10 null transgenic mice have increased cellular proliferation mediated by the overexpression of c-myc (Reichelt & Magin, 2002). In our present study, reduction of KRT10 is evident in both BQ chewers and smokers but the reduction is more significant in the BQ chewers suggesting that suppression of KRT10 may be more important in oral carcinogenesis induced by BQ in comparison to those caused by smoking. Similarly, a difference in KRT10 was observed between oral cancer patients from Sudan and those from Norway (Ibrahim et al., 2003).

### CONCLUSION

The aim of many microarray experiments is to determine the global genetic alterations that distinguish cancer cells from their normal counterparts, which in turn promotes an understanding of the gene expression changes that are important for cancer development and progression. More importantly, the technique may lead to the identification of biomarkers that could be used in the identification of cancer cells, as clinical prognostic indicators or as therapeutic targets. Significantly, biomarkers are already in use clinically for the routine assessment of the prognosis in new patients with breast cancer based on its association with tumour relapse (Duffy, 2002; Hundsdorfer et al, 2005).

The genes that are differentially expressed between OSCC and normal oral mucosa have been identified in the present invention. These genes may contribute to oral carcinogenesis by promoting cell proliferation and maintaining the pluripotent state of the cells that ultimately drive cancer development. There are gene expression differences between oral cancers with differing risk factors.

1-30. (canceled)

31. A method for aiding assessment of a patient's risk of developing oral and nasopharyngeal cancer, or likely severity or likelihood of progression of oral and nasopharyngeal cancer, or aiding in selection of a cancer treatment regime for the patient in treating oral and nasopharyngeal cancers, or aiding in assessment of a cancer treatment regime for treating oral and nasopharyngeal cancers, or the detection and diagnosis of oral and nasopharyngeal cancers, the method comprising:
(a) determining in a biological sample from the patient the amount of the expression level of GNA-12; and
(b) comparing the determined expression level of said gene in said biological sample with the level in a reference.

32. The method according to claim 31, wherein step (a) further comprising the step of determining the amount of the expression level of IFITM3.

33. The method according to claim 31, wherein said expression level is the RNA expression level of GNAl2 or IFITM3 in said biological sample.

34. The method according to claim 31, further comprising determining the RNA levels of GNA-12 or IFITM3 in the sample.

35. The method according to claim 33, wherein the RNA levels of GNA-12 or IFITM3 are determined by the use of at least two oligonucleotide primers, the primers selected from Table 2.

36. The method according to claim 31, wherein said expression level is the protein expression level of GNAl2 or IFITM3 in said biological sample.

37. The method according to claim 31, further comprising determining the protein levels of GNAl2 or IFITM3 in the sample.

38. The method according to claim 31, wherein the reference is normal tissue.

39. The method according to claim 31, wherein the biological sample is selected from the group consising of oral mucosal tissue, nasopharyngeal swab and mouth washing.

40. A method of detecting oral and nasopharyngeal cancers in a patient, the method comprising administering to the patient an anti-GNA-12 antibody or a fragment or derivative thereof labelled with a detectable label, allowing the labelled antibody to locate to the cancer, and imaging the cancer.

41. Use of a nucleic acid which selectively hybridises to GNA-12 mRNA in the manufacture of a reagent for diagnosing oral and nasopharyngeal cancers.

42. Use of a nucleic acid according to claim 41 in a method of diagnosing oral and nasopharyngeal cancers.

43. Use of a molecule which selectively binds to GNA-12 polypeptide or a natural fragment or variant thereof in the manufacture of a reagent for diagnosing or imaging oral and nasopharyngeal cancers.

44. A method of treating oral and nasopharyngeal cancers, the method comprising administering to the patient an effective amount of GNA-12 polypeptide or a variant or fusion or fragment thereof, or an effective amount of a nucleic acid encoding a GNA-12 polypeptide or a variant or fragment or fusion thereof, wherein the amount of said polypeptide or amount of said nucleic acid is effective to provoke an anti-cancer cell immune response in said patient.

45. Use of an effective amount of GNA-12 polypeptide or a variant or fusion or fragment thereof, or an effective amount of a nucleic acid encoding a GNA-12 polypeptide or a variant or fragment or fusion thereof, in the manufacture of a medicament for treating oral and nasopharyngeal cancers.

46. A oral and nasopharyngeal cancer vaccine comprising a GNA-12 polypeptide or variant or fragment thereof, or a nucleic acid encoding GNA-12 polypeptide or fragment or variant thereof.

47. A method of treating oral and nasopharyngeal cancers in a patient, the method comprising administering to the patient a molecule that modulates the activity of the GNA-12 gene or their products.

48. The method according to claim 47, wherein the molecule that modulates the activity of the GNA-12 gene or their products is a GNA-12 antisense agent, siRNA or antibody.

49. Use of a molecule that modulates the activity of the GNA-12 gene or their products in the manufacture of a medicament for treating oral and nasopharyngeal cancers.

50. A method of treating oral and nasopharyngeal cancers in a patient, the method comprising administering to the patient an antibody directed to GNAl2.

51. Use of an antibody directed to GNA-12 in the manufacture of a medicament for the treating oral and nasopharyngeal cancers.

52. The method according to claim 50, wherein the antibody is labelled with a directly or indirectly cytotoxic agent.

53. The method according to claim 52, wherein the cytotoxic agent is a directly cytotoxic chemotherapeutic agent.

54. The method according to claim 52, wherein the cytotoxic agent is a directly cytotoxic polypeptide.

55. The method according to claim 52, wherein the cytotoxic agent is an agent which is able to convert a relatively non-toxic prodrug into a cytotoxic drug.

56. The method according to claim 52, wherein the cytotoxic agent is a radiosensitizer.

57. A kit for aiding assessment of a patient's risk of developing oral and nasopharyngeal cancers, or likely severity or likelihood of progression of oral and nasopharyngeal cancers, or aiding in selection of a cancer treatment regime for the patient in treating oral and nasopharyngeal cancers, or aiding in assessment of a cancer treatment regime for treating oral and nasopharyngeal cancers, the kit comprising at least one reagent for determing the amount of the expression level of GNA-12, and a package insert containing instructions using the kit.

58. The kit according to claim 57, further comprising a reagent for determining the amount of the expression level of IFITM3.

59. The kit according to claim 57, wherein the reagents for the detection are one or more oligonucleotide primers.

60. The kit according to claim 59, wherein the one or more oligonucleotide primers comprise, consist essentially of or consist of any one of the nucleotide sequences in Table 2.

61. The kit according to claim 57, wherein the kit is an array or chip.


SEQUENCE LISTING (26 sequences)

SEQ ID NO: 1 - DNA, 4398 bp, Homo sapiens
gggcgacgagtgcgggcctcggagcgactgcagcggcggcggcggacgcggcctgaggcgagcggcggggcgtggggcggtgcctcggcccgggctcgccctcgccggcgggagcgtccatggcccccgggcgccggcggggcgcggccgcggcctgaggggccatgtccggggtggtgcggaccctcagccgctgcctgctgccggccgaggccggcggggcccgcgagcgcagggcgggcagcggcgcgcgcgacgcggagcgcgaggcccggaggcgtagccgcgacatcgacgcgctgctggcccgcgagcggcgcgcggtccggcgcctggtgaagatcctgctgctgggcgcgggcgagagcggcaagtccacgttcctcaagcagatgcgcatcatccacggccgcgagttcgaccagaaggcgctgctggagttccgcgacaccatcttcgacaacatcctcaagggctcaagggttcttgttgatgcacgagataagcttggcattccttggcagtattctgaaaatgagaagcatgggatgttcctgatggccttcgagaacaaggcggggctgcctgtggagccggccaccttccagctgtacgtcccggccctgagcgcactctggagggattctggcatcagggaggctttcagccggagaagcgagtttcagctgggggagtcggtgaagtacttcctggacaacttggaccggatcggccagctgaattactttcctagtaagcaagatatcctgctggctaggaaagccaccaagggaattgtggagcatgacttcgttattaagaagatcccctttaagatggtggatgtgggcggccagcggtcccagcgccagaagtggttccagtgcttcgacgggatcacgtccatcctgttcatggtctcctccagcgagtacgaccaggtcctcatggaggacaggcgcaccaaccggctggtggagtccatgaacatcttcgagaccatcgtcaacaacaagctcttcttcaacgtctccatcattctcttcctcaacaagatggacctcctggtggagaaggtgaagaccgtgagcatcaagaagcacttcccggacttcaggggcgacccgcacaggctggaggacgtccagcgctacctggtccagtgcttcgacaggaagagacggaaccgcagcaagccactcttccaccacttcaccaccgccatcgacaccgagaacgtccgcttcgtgttccatgctgtgaaagacaccatcctgcaggagaacctgaaggacatcatgctgcagtgagcgaggaagccccggggtttgtcgtcgttgagcagcccccacggctgtcggtcagactcttgggtgtgtgttgtctgtgtggtccttgagtgggtttctcggatccgtgccctggaatacctggctcaggaatgctgtcagaccagccagccagcgagctctaggcaaaaggacatggaaactgtcacgttagctactgaatcctgggggcgagtgaaactactgaaaatccgagtgatgatgttgtgaatacggaacacctaatcacacagcttgctttgcttttacagaaacgttcctctttttctgacgcagtttaattgaggaccgtgttgtgtgtgtatgtgtgtacacacgctctgtctttaatgacagaaacacaaaaaccagctggccttgcagacggcttttctaactcacaagtcttccctgagacagactaacctgaaagctttgcctaacagtagcttgtagagatccagtgcacgccgatgctgctaaactcagtgcctgagcccggccctgcagccccagccgcagtgtctgaaggccacctcccaaagggagcacgttgccttttcaaactcccgtgccgatttcctaagagcccctagtccaagcctctcagatgaagctgaggagccgtgcctaggatcccttcccagctctgaggacgggctgcagagctctgcaggtgtggattcaccttacgcccctacagcaggctcagcccttcccaccctgccccatgcccagcagcacaacacggagtgagacaggatgcccacggtgactgccgctccgtccgtgcacacacagcggtgctcttctccccttagccacccactgcccaacccaacggcaaagacacagaaaccaggtccccttgcagacggctctcccatcttcctgcaagtcatctgctcacacacagttggcagcacatagcgtttccttctttcagaaacattcctcttctggggcttcagaaagctggcaaggccactagcagagcttttgttaatgccccagctgcttggcgagctaacagctgacctttcgggaagcccacagacgctggaggaatcttgagtttctccaaactgccgctccaccagtgcctttggacagccgtgcctgttcgccgctctccctaagtctgattctcatcgaggcccctcgcttctatgactgtgcttgcagaagagtaaacactctcggatgccgctgtcctgggggagcccgcgggagcctgtgaatgttgatacgagctggccagtcctgggcccagctcacttgtccagctacctgccaggtggctttcactgtgtttaaaatacattgcattccaagctggtcccctctgtgtatcactctactgagaaatcctgcctagtgtgttttgggatgtgtcctagcatttacaagaaaatgaaaagcgtcctcttaattggcacccgaatgttgctgtggctcagtcacatatcccagggccctcgtcccgaggccgtgctgccccgagccccgagcccctctgcagctcacccttggcttgttttccgcaaacccggtaaacgcaagcccttggggcagatgcagaagcagaagagggaggggaaacctgcctctgggtcaccctgttagcacagcgttctcatcgggagacagcatggaactctctctcgcagtgctcgaggctgtgtgtcagtgtttgctgggcttgtggctccttttttggctggataaagaagtcgctgtttttgtactgcttctgtggctcttcacagacctcacggatgtgaccggagatgagtgccgatgaccacgttttaaaggagaaagagagctcctggtggggccctcggggtggtctcaggtcccatttgcagtctgcaacagtgacgcgcagcccggtccggagcgtggtgagctttgtttgccttctgggtcagctttcgctgtgtctcctgtgtgtgttagaatccagagcccagaggaagtgcaagcgggtcctccgccaacggggagagcctcttcgcggcgctgttggcgacagcagcgctgtgattcgcgtagcaggggagttgtttgaaacaccttcctgagtagtccggccttgtcaatgagtgcttgttttcctttaaacagtctgacatatttactcgtcactttcaaaccagaagcatgagaggaaggagatattgtggggtccgtttaactcgatagaaagcgcagggggatggcccccggcgcgggctcttgacccgctcagcgctgaccccaccgccctggccgaggcacttggccttgctgagctggacttcctcctcctcctcctcatgaccggggtgaattagaacgtttttaaagacacccccttccaaattctgtaacacattgtaattggagaagaaggaaactctgcaaggctaaactgtcattcacaacttggctacacatagactctagtcagttttgtctccagaaccttaggcttttgtattttttaattttaatttcactgttaatccttattgtcttttttattaagatgttggaaaagcaggaggtagttgtgcctcaattattgcaaaaatgtaacaataaagttcctcaaaataagatctgttcctcatagctatactgtgtacacataagacgcatatagggttttactgaaatctatttttaactcttatgttcgtagagaaattgtttcaaggattttgagtcataggtctgtaatttatagagatctctagaattcttattgtaattttcctacttctttgataaaagaaaaataagtcagattgttaactccaagattgaaaaaaaaaactcttgaaagaagattattagttgtaactaatttaggggttctgggcacagacatctaacctggtattgtaaggcagaggctcccattggaatggtagtggtccgggtcagttgttcatggtgtaagctttgcacagtgtattaacattgggagggtctggcttgaaaatttggccaccctcagcctctgaatgtttattaaaataaatttagtctttctttgcttaatataaaaaaaaaaaaaaaa

SEQ ID NO: 2 - protein, 381 aa, Homo sapiens
MSGVVRTLSRCLLPAEAGGARERRAGSGARDAEREARRRSRDIDALLARERRAVRRLVKILLLGAGESGKSTFLKQMRIIHGREFDQKALLEFRDTIFDNILKGSRVLVDARDKLGIPWQYSENEKHGMFLMAFENKAGLPVEPATFQLYVPALSALWRDSGIREAFSRRSEFQLGESVKYFLDNLDRIGQLNYFPSKQDILLARKATKGIVEHDFVIKKIPFKMVDVGGQRSQRQKWFQCFDGITSILFMVSSSEYDQVLMEDRRTNRLVESMNIFETIVNNKLFFNVSIILFLNKMDLLVEKVKTVSIKKHFPDFRGDPHRLEDVQRYLVQCFDRKRRNRSKPLFHHFTTAIDTENVRFVFHAVKDTILQENLKDIMLQ

SEQ ID NO: 3 - DNA, 678 bp, Homo sapiens
aggaaaaggaaactgttgagaaaccgaaactactggggaaagggagggctcactgagaaccatcccagtaacccgaccgccgctggtcttcgctggacaccatgaatcacactgtccaaaccttcttctctcctgtcaacagtggccagccccccaactatgagatgctcaaggaggagcacgaggtggctgtgctgggggcgccccacaaccctgctcccccgacgtccaccgtgatccacatccgcagcgagacctccgtgcccgaccatgtcgtctggtccctgttcaacaccctcttcatgaacccctgctgcctgggcttcatagcattcgcctactccgtgaagtctagggacaggaagatggttggcgacgtgaccggggcccaggcctatgcctccaccgccaagtgcctgaacatctgggccctgattctgggcatcctcatgaccattctgctcatcgtcatcccagtgctgatcttccaggcctatggatagatcaggaggcatcactgaggccaggagctctgcccatgacctgtatcccacgtactccaacttccattcctcgccctgcccccggagccgagtcctgtatcagccctttatcctcacacgcttttctacaatggcattcaataaagtgcacgtgtttctggtgctaaaaaaaaaa

SEQ ID NO: 4 - protein, 133 aa, Homo sapiens
MNHTVQTFFSPVNSGQPPNYEMLKEEHEVAVLGAPHNPAPPTSTVIHIRSETSVPDHVVWSLFNTLFMNPCCLGFIAFAYSVKSRDRKMVGDVTGAQAYASTAKCLNIWALILGILMTILLIVIPVLIFQAYG

SEQ ID NO: 5 - DNA, 20 bp, Artificial Sequence
  Synthetic oligonucleotide
tgggaatgctaccaaagcct

SEQ ID NO: 6 - DNA, 20 bp, Artificial Sequence
  Synthetic oligonucleotide
tcatgcaggtgaggaacgag

SEQ ID NO: 7 - DNA, 20 bp, Artificial Sequence
  Synthetic oligonucleotide
tgtggaccatgccattgaga

SEQ ID NO: 8 - DNA, 20 bp, Artificial Sequence
  Synthetic oligonucleotide
tgagcatcccctccaatacc

SEQ ID NO: 9 - DNA, 20 bp, Artificial Sequence
  Synthetic oligonucleotide
ggcagggctctgatattcca

SEQ ID NO: 10 - DNA, 21 bp, Artificial Sequence
  Synthetic oligonucleotide
ggccccaggagtgacctataa

SEQ ID NO: 11 - DNA, 29 bp, Artificial Sequence
  Synthetic oligonucleotide
ataagtcagattgttaactccaagattga

SEQ ID NO: 12 - DNA, 20 bp, Artificial Sequence
  Synthetic oligonucleotide
agccagaccctcccaatgtt

SEQ ID NO: 13 - DNA, 22 bp, Artificial Sequence
  Synthetic oligonucleotide
gaccattctgctcatcgtcatc

SEQ ID NO: 14 - DNA, 25 bp, Artificial Sequence
  Synthetic oligonucleotide
gcactttattgaatgccattgtaga

SEQ ID NO: 15 - DNA, 18 bp, Artificial Sequence
  Synthetic oligonucleotide
ccagaatcagaaccgaga

SEQ ID NO: 16 - DNA, 18 bp, Artificial Sequence
  Synthetic oligonucleotide
ccaaaatctccgtcctca

SEQ ID NO: 17 - DNA, 22 bp, Artificial Sequence
  Synthetic oligonucleotide
tcggttagtggaagcatgattg

SEQ ID NO: 18 - DNA, 20 bp, Artificial Sequence
  Synthetic oligonucleotide
agacgttcgggtgggatctc

SEQ ID NO: 19 - DNA, 19 bp, Artificial Sequence
  Synthetic oligonucleotide
gccgctacgagggtcagtt

SEQ ID NO: 20 - DNA, 21 bp, Artificial Sequence
  Synthetic oligonucleotide
tccattacagatgcccccatt

SEQ ID NO: 21 - DNA, 21 bp, Artificial Sequence
  Synthetic oligonucleotide
gactgcccagattcccacact

SEQ ID NO: 22 - DNA, 20 bp, Artificial Sequence
  Synthetic oligonucleotide
acgatggagaccaccaccaa

SEQ ID NO: 23 - DNA, 21 bp, Artificial Sequence
  Synthetic oligonucleotide
aggccacaagtcctcctcttc

SEQ ID NO: 24 - DNA, 29 bp, Artificial Sequence
  Synthetic oligonucleotide
tttcctcttgatgcagtttaatagtagtg

SEQ ID NO: 25 - DNA, 19 bp, Artificial Sequence
  Synthetic oligonucleotide
gaaggtgaaggtcggagtc

SEQ ID NO: 26 - DNA, 17 bp, Artificial Sequence
  Synthetic oligonucleotide
gaagatggtgggatttc